(19) United States
(12) Patent Application Publication
(10) Pub. No.: US 2019/0080048 A1
(43) Pub. Date: Mar. 14, 2019

(54) SYSTEM AND METHOD FOR PROCESSING GENOTYPE INFORMATION RELATING TO OPIOID RESPONSE

(71) Applicant: PROOVE BIOSCIENCES, INC., Irvine, CA (US)

(72) Inventor: Brian MESHKIN, Ladera Ranch, CA (US)

(21) Appl. No.: 15/570,312

(22) PCT Filed: Apr. 28, 2016
(86) PCT No.: PCT/US2016/029827

§ 371 Date: Nov. 2, 2018

(51) Int. Cl. G16H 50/20; C12Q 1/6858; C12Q 1/6883; C12Q 1/6827

(52) U.S. Cl. CPC G06F 19/22; G16H 50/20; C12Q 1/6858; C12Q 2600/156; C12Q 1/6827; C12Q 2600/172; C12Q 2600/106; C12Q 1/6883

(57) ABSTRACT

There are systems and methods for preparing or using prognostic information about opioid response. The information may include determining patient information, including DNA information, associated with a human subject; determining from the DNA information whether a subject genotype of the human subject includes one or more SNP diploid polymorphisms by detecting, utilizing a detection technology and the DNA information, a presence or absence of the one or more SNP diploid polymorphisms in the subject genotype, wherein each SNP diploid polymorphism of the one or more SNP diploid polymorphisms includes a combination of two SNP alleles associated with one SNP location; and determining an opioid response associated with the human subject based, at least in part, on the presence or absence of the one or more SNP diploid polymorphisms in the subject genotype.

### PRIORITY

This application claims priority to U.S. Provisional Application No. 62/153,639 entitled “System and Method for Processing Genotype Information Relating to Opioid Response” by Brian Meshkin filed on Apr. 28, 2015, which is incorporated herein by reference in its entirety.

### SEQUENCE LISTING

A sequence listing submitted in computer readable format is hereby incorporated by reference. The computer readable file is named OPIOIDRESPONSE-PCT_P8283PC00_SL.txt, is 4.49 KB in size, and was created on Jun. 6, 2016.

### BACKGROUND

In nature, organisms of the same species usually differ from each other in various aspects such as in their appearance or in one or more aspects of their biology. The differences are often based on genetic distinctions, some of which are called polymorphisms. Polymorphisms are often observed at the level of the whole individual (i.e., phenotype polymorphism), in variant forms of proteins and blood group substances (i.e., biochemical polymorphism), in morphological features of chromosomes (i.e., chromosomal polymorphism), and at the level of DNA in differences of nucleotides and/or nucleotide sequences (i.e., genetic polymorphism).

Examples of genetic polymorphisms include alleles and haplotypes. An allele is an alternative form of a gene, such as one member of a pair, that is located at a specific position on a chromosome and are known as single nucleotide polymorphisms (SNPs). A haplotype is a combination of alleles, or a combination of SNPs on the same chromosome. An example of a genetic polymorphism is an occurrence of one or more genetically alternative phenotypes in a subject due to the presence or absence of an allele or haplotype.

Genetic polymorphisms can play a role in determining differences in an individual's response to a species of drug, a drug dosage or a therapy including one drug or a combination of drugs. Pharmacogenetics and pharmacogenomics are multidisciplinary research efforts to study the relationships among genotypes, gene expression profiles, and phenotypes, as often expressed through the variability between individuals in response to the drugs taken. Since the initial sequencing of the human genome, more than a million SNPs have been identified. Some of these SNPs have been used to predict clinical predispositions or responses based upon data gathered from pharmacogenomic studies.

Chronic pain affects up to 100 million Americans (more than heart disease, cancer, and diabetes combined) and has clinical and public health implications. Opioid drugs, such as hydrocodone, hydromorphone, morphine, oxycodone, tramadol, and the like, while being effective for treating and relieving pain in some individuals, often to not provide an effective response in others. The use of opioid medications has increased exponentially in the last two decades. In particular, most prescriptions for opioids have been for hydrocodone and oxycodone. Most patients are not able to control their pain with their current medications, so doctors increase the dose, potency, or class of pain medications. However, responses to opioid medications display considerable individual variability and the assessing the likelihood of an individual's response to opioids continue to be a challenge.

While opioids represent one of the most frequently prescribed class of drugs, inadequate prescribing practices remain frequent. It is known that first-line or maintenance opioid medications are effective for some patients, but not others—even in instances of similar mechanisms of injury and/or etiologies of pain. However the mechanism for these differences remains somewhat unclear. Emerging scientific evidence suggests that genetic variants may play a part. Genetic factors overall are believed to account for 20% to 95% of the observed variations in drug response in individuals. In pharmacogenomics, there is a desire to identify new polymorphisms and haplotypes associated with opioid response in patients who are candidates for or who are taking opioid medications. The genotype information of a patient may help a prescriber understand whether the patient is at risk for a poor response to various opioid medications.

A patient's genotype information is often utilized to help a prescriber decide between medications based on information associated with a patient's genetic profile (i.e., genotype information). There is a desire to utilize a patient's genotype information in determining the patient's predisposition to opioid response. There is also a desire for methods for predicting and/or diagnosing individuals exhibiting irregular predispositions to opioid response. Furthermore, there is also a desire to determine genetic information, such as polymorphisms, which may be utilized for predicting variations in opioid response among individuals. There is also a desire to implement systems processing and distribing the detected genetic information in a systematic way. Such genetic information would be useful in providing prognostic information about treatment options for a patient.

Although it is known generally that opioid response may be associated with genetics—a factor not routinely considered, there is no rigourous methodology to systematically provide doctors with an ability to identify patients who may misuse and/or have a genetic predisposition for poor opioid response. Such systems and methods would be beneficial to provide information that improves accuracy in identifying patients at risk for poor opioid response.

Given the foregoing, and to address the above-described limitations, systems and methods are desired for identifying, estimating and/or determining a potential for success of an individual patient's clinical outcome in response to being prescribed an opioid medication.

### SUMMARY

This summary is provided to introduce a selection of concepts that are further described in the Detailed Description below. The genes, polymorphisms, sequences and sequence identifiers (i.e., SEQ IDs or SEQ ID Numbers) listed or referenced herein are also described in greater detail below in the Detailed Description. This summary is not intended to identify key or essential features of the claimed subject matter. Also, this summary is not intended as an aid in determining the scope of the claimed subject matter.

The present invention meets the above-identified needs by providing systems, methods and computer readable mediums (CRMs) for preparing and utilizing prognostic information associated with a predisposition to poor opioid response in a patient. The prognostic information is derived from genotype information about a patient's gene profile. The genotype information may be obtained by, inter alia, assaying a sample of genetic material associated with a patient.

The systems, methods and CRMs, according to the principles of the invention, can be utilized to determine prognostic information associated with opioid response based on the patient's opioid predisposition. The prognostic information may be used for addressing prescription needs directed to caring for an individual patient. It may also be utilized in managing large healthcare entities, such as insurance providers, utilizing comprehensive business intelligence systems. These and other objects are accomplished by systems, methods and CRMs directed to preparing and utilizing prognostic information associated with opioid response predisposition in a patient, in accordance with the principles of the invention.

According to a first principal of the invention, there is a method. The method may include facilitating a processing of and/or processing (1) data and/or (2) information and/or (3) at least one signal, the (1) data and/or (2) information and/or (3) at least one signal based, at least in part, on any combination of at least part of the following: determining patient information, including DNA information, associated with a human subject; determining from the DNA information whether a subject genotype of the human subject includes one or more SNP diploid polymorphisms by detecting, utilizing a detection technology and the DNA information, a presence or absence of the one or more SNP diploid polymorphisms in the subject genotype, wherein each SNP diploid polymorphism of the one or more SNP diploid polymorphisms includes a combination of two SNP alleles associated with one SNP location, wherein the one or more SNP diploid polymorphisms are selected from the SNP diploid group: OPRM1-ANC, OPRM1-HET, and OPRM1-NONA in the OPRM1 gene, SLC6A3-ANC, SLC6A3-HET, and SLC6A3-NONA in the SLC6A3 gene, COMT-ANC, COMT-HET, and COMT-NONA in the COMT gene, ABCB1(C3435T)-ANC, ABCB1(C3435T)-HET, and ABCB1(C3435T)-NONA in the ABCB1 gene, ABCB1(C1236T)-ANC, ABCB1(C1236T)-HET, and ABCB1(C1236T)-NONA in the ABCB1 gene, ABCB1(G2677A/T)-ANC, ABCB1(G2677A/T)-HET, and ABCB1(G2677A/T)-NONA in the ABCB1 gene, SLC22A1 OCT A>C-ANC, SLC22A 1_OCT A>C-HET, and SLC22A 1_OCT A>C-NONA in the SLC22A 1_OCT A>C-gene, HTR2A-ANC, HT2RA-HET, and HT2RA-NONA in the HT2RA gene, GABRG2-ANC, GABRG2-HET, and GABRG2-NONA in the GABRG2 gene, and SLC6A4-ANC, SLC6A4-HET, and SLC6A4-NONA in the SLC6A4 gene; and determining an opioid response associated with the human subject based, at least in part, on the presence or absence of the one or more SNP diploid polymorphisms in the subject genotype.

The method may also include wherein the (1) data and/or (2) information and/or (3) at least one signal are further based, at least in part, on any combination of the following: determining from the DNA information whether a subject genotype of the human subject includes at least two CYP haplotype polymorphisms by detecting, utilizing a detection technology and the DNA information, a presence or absence of the at least two CYP haplotype polymorphisms in the subject genotype, wherein at least one or more CYP haplotype polymorphisms are selected from CYP3A4 or CYP3A5 star alleles, wherein at least one or more CYP haplotype polymorphisms are selected from CYP2D6 star alleles, wherein the method for determining the opioid response associated with the human subject, is an ex vivo method.

The method may also include determining a comparing of a region, including the one or more SNP diploid polymorphisms, of the subject genotype with a corresponding region of a predetermined reference genotype, wherein characteristics of the corresponding region of the reference genotype are based upon a predetermined population norm; determining prognostic information associated with the human subject based on the determined opioid response; and determining a therapy for the human subject based on the determined prognostic information associated with the human subject, wherein the one or more SNP diploid polymorphisms include at least any number from two through ten SNP diploid polymorphisms from the SNP diploid group.

According to a second principle of the invention, there is an apparatus. The apparatus may include any combination of at least one processor; and at least one memory including computer program code for one or more programs, the at least one memory and the computer program code configured to, with the at least one processor, cause the apparatus to perform at least the following, determine patient information, including DNA information, associated with a human subject; determine from the DNA information whether a subject genotype of the human subject includes one or more SNP diploid polymorphisms by detecting, utilizing a detection technology and the DNA information, a presence or absence of the one or more SNP diploid polymorphisms in the subject genotype, wherein each SNP diploid polymorphism of the one or more SNP diploid polymorphisms includes a combination of two SNP alleles associated with one SNP location, wherein the one or more SNP diploid polymorphisms are selected from the SNP diploid group: OPRM1-ANC, OPRM1-HET, and OPRM1-NONA in the OPRM1 gene, SLC6A3-ANC, SLC6A3-HET, and SLC6A3-NONA in the SLC6A3 gene, COMT-ANC, COMT-HET, and COMT-NONA in the COMT gene, ABCB1(C3435T)-ANC, ABCB1(C3435T)-HET, and ABCB1(C3435T)-NONA in the ABCB1 gene, ABCB1(C1236T)-ANC, ABCB1(C1236T)-HET, and ABCB1(C1236T)-NONA in the ABCB1 gene, ABCB1(G2677A/T)-ANC, ABCB1(G2677A/T)-HET, and ABCB1(G2677A/T)-NONA in the ABCB1 gene, SLC22A1 OCT A>C-ANC, SLC22A 1_OCT A>C-HET, and SLC22A 1_OCT A>C-NONA in the SLC22A 1_OCT A>C-gene, HTR2A-ANC, HT2RA-HET, and HT2RA-NONA in the HT2RA gene, GABRG2-ANC, GABRG2-HET, and GABRG2-NONA in the GABRG2 gene, and SLC6A4-ANC, SLC6A4-HET, and SLC6A4-NONA in the SLC6A4 gene; determine an opioid response associated with the human subject based, at least in part, on the presence or absence of the one or more SNP diploid polymorphisms in the subject genotype.

According to a third principle of the invention, there is a non-transitory computer readable medium. The medium may store any combination of computer readable instructions that when executed by at least one processor perform a method, the method comprising facilitating a processing of and/or processing (1) data and/or (2) information and/or (3) at least one signal, the (1) data and/or (2) information and/or (3) at least one signal based, at least in part, on any combination of at least part of the following: determining patient information, including DNA information, associated with a human subject; determining from the DNA information whether a subject genotype of the human subject includes one or more SNP diploid polymorphisms by detecting, utilizing a detection technology and the DNA information, a presence or absence of the one or more SNP diploid polymorphisms in the subject genotype, wherein each SNP diploid polymorphism of the one or more SNP diploid polymorphisms includes a combination of two SNP alleles associated with one SNP location, wherein the one or more SNP diploid polymorphisms are selected from the SNP diploid group: OPRM1-ANC, OPRM1-HET, and OPRM1-NONA in the OPRM1 gene, SLC6A3-ANC, SLC6A3-HET, and SLC6A3-NONA in the SLC6A3 gene, COMT-ANC, COMT-HET, and COMT-NONA in the COMT gene, ABCB1(C3435T)-ANC, ABCB1(C3435T)-HET, and ABCB1(C3435T)-NONA in the ABCB1 gene, ABCB1(C1236T)-ANC, ABCB1(C1236T)-HET, and ABCB1(C1236T)-NONA in the ABCB1 gene, ABCB1(G2677A/T)-ANC, ABCB1(G2677A/T)-HET, and ABCB1(G2677A/T)-NONA in the ABCB1 gene, SLC22A1 OCT A>C-ANC, SLC22A 1_OCT A>C-HET, and SLC22A 1_OCT A>C-NONA in the SLC22A 1_OCT A>C-gene, HTR2A-ANC, HT2RA-HET, and HT2RA-NONA in the HT2RA gene, GABRG2-ANC, GABRG2-HET, and GABRG2-NONA in the GABRG2 gene, and SLC6A4-ANC, SLC6A4-HET, and SLC6A4-NONA in the SLC6A4 gene; and determining an opioid response associated with the human subject based, at least in part, on the presence or absence of the one or more SNP diploid polymorphisms in the subject genotype.

The above summary is not intended to describe each embodiment or every implementation of the present invention. Further features, their nature and various advantages are made more apparent from the accompanying drawings and the following examples and embodiments.

### BRIEF DESCRIPTION OF THE DRAWINGS

Features and advantages of the present invention become more apparent from the detailed description, set forth below, when taken in conjunction with the drawings. In the drawings, like reference numbers indicate identical or functionally similar elements. Additionally, a left-most digit of a reference number identifies a drawing in which the reference number first appears. In addition, it should be understood that the drawings in the figures which highlight an aspect, methodology, functionality and/or advantage of the present invention, are presented for example purposes only. The present invention is sufficiently flexible such that it may be implemented in ways other than shown in the accompanying figures.

FIG. 1 is a block diagram illustrating an assay system which may be utilized for preparing genotype information from a sample of genetic material, according to an example;

FIG. 2 is a block diagram illustrating a prognostic information system which may be utilized for preparing and/or utilizing prognostic information utilizing the genotype information prepared using the assay system of FIG. 1, according to an example;

FIG. 3 is a flow diagram illustrating a prognostic information process for identifying a risk to a patient utilizing the assay system of FIG. 1 and the prognostic information system of FIG. 2, according to an example; and

FIG. 4 is a block diagram illustrating a computer system providing a platform for the assay system of FIG. 1 or the prognostic information system of FIG. 2, according to various examples.

### DETAILED DESCRIPTION

The present invention is useful for preparing and/or utilizing prognostic information about a patient. The prognostic information may be utilized to determine an appropriate therapy for the patient based on their genotype and phenotype information and identify their genetic predisposition to opioid therapies. The genetic predisposition may be associated with the selection of an opioid medication, a dosage of the opioid medication and the utilization of the opioid medication in a regimen for treating the patient's medical condition.

The prognostic information may also be utilized for determining dose adjustments that may help a prescriber understand why a patient is or is not responding to an opioid medication dosage, such as an “average” dose. The prognostic information may also be utilized by a prescriber to decide between medications based on the patient's genetic predisposition to opioid response. The prognostic information may also be utilized for predicting and/or diagnosing individuals exhibiting a regular or irregular predisposition to opioid response. Such genetic information can be very useful in providing prognostic information about treatment options for a patient. The patient may be associated with a medical condition. The patient may also have already been prescribed a medication for treating the medical condition. The present invention has been found to be advantageous for determining a treatment for a patient who may have a regular or irregular predisposition to opioid response. While the present invention is not necessarily limited to such applications, various aspects of the invention may be appreciated through a discussion of the various examples in this context, as illustrated through the examples below.

For simplicity and illustrative purposes, the present invention is described by referring mainly to embodiments, principles and examples thereof. In the following description, numerous specific details are set forth in order to provide a thorough understanding of the examples. It is readily apparent however, that the embodiments may be practiced without limitation to these specific details. In other instances, some embodiments have not been described in detail so as not to unnecessarily obscure the description. Furthermore, different embodiments are described below. The embodiments may be used or performed together in different combinations.

The operation and effects of certain embodiments can be more fully appreciated from the examples described below. The embodiments on which these examples are based are representative only. The selection of embodiments is to illustrate the principles of the invention and does not indicate that variables, functions, conditions, techniques, configurations and designs, etc., which are not described in the examples are not suitable for use, or that subject matter not described in the examples is excluded from the scope of the appended claims and their equivalents. The significance of the examples can be better understood by comparing the results obtained therefrom with potential results which can be obtained from tests or trials that may be or may have been designed to serve as controlled experiments and provide a basis for comparison.

Before the systems and methods are described, it is understood that the invention is not limited to the particular methodologies, protocols, systems, platforms, assays, and the like which are described, as these may vary. It is also to be understood that the terminology used herein is intended to describe particular embodiments of the present invention, and is in no way intended to limit the scope of the present invention as set forth in the appended claims and their equivalents.

Throughout this disclosure, various publications, such as patents and published patent specifications, are referenced by an identifying citation. The disclosures of these publications are hereby incorporated by reference in their entirety into the present disclosure in order to more fully describe the state of the art to which the invention pertains.

The practice of the present invention employs, unless otherwise indicated, conventional techniques of molecular biology, microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3rd edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5th edition (2005)); OLIGONUCLEOTIDE SYNTHESIS (M. J. Gait ed. (1984)); Mullis et al., U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)); MANIPULATING THE MOUSE EMBRYO: A LABORATORY MANUAL 3rd edition (Cold Spring Harbor Laboratory Press, Cold Spring Harbor, N.Y. (2002)).

### Definitions

As used herein, certain terms have the following defined meanings. As used herein, the singular form “a,” “an” and “the” includes the singular and plural references unless the context clearly dictates otherwise. For example, the term “a cell” includes a single cell and a plurality of cells, including mixtures thereof.

As used herein, the terms “based on,” “comprises,” “comprising,” “includes,” “including,” “has,” “having” or any other variation thereof, are intended to cover a non-exclusive inclusion. For example, a system, process, method, article, or apparatus that comprises a list of elements is not necessarily limited to only those elements but may include other elements not expressly listed or inherent to such system, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B is true (or present).

All numerical designations, e.g., pH, temperature, time, concentration, and molecular weight, including ranges, are approximations which may be varied (+) or (−) by minor increments, such as, of 0.1. It is to be understood, although not always explicitly stated, that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known to those of ordinary skill in the art.

The term “allele” which is used interchangeably herein with the term “allelic variant” refers to alternative forms of a gene or any portions thereof. Alleles may occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene can differ from each other in a single nucleotide, or several nucleotides, and can include substitutions, deletions and insertions of nucleotides. An allele of a gene can also be an ancestral form of a gene or a form of a gene containing a mutation.

The term “haplotype” refers to a combination of alleles on a chromosome or a combination of SNPs within an allele on one chromosome. The alleles or SNPs may or may not be at adjacent locations (loci) on a chromosome. A haplotype may be at one locus, at several loci or an entire chromosome.

The term “ancestral,” when applied to describe an allele in a human, refers to an allele of a gene that is the same or nearest to a corresponding allele appearing in the corresponding gene of the chimpanzee genome. Often, but not always, a human ancestral allele is the most prevalent human allelic variant appearing in nature—i.e., the allele with the highest gene frequency in a population of the human species.

The term “wild-type,” when applied to describe an allele, refers to an allele of a gene which, when it is present in two copies in a subject, results in a wild-type phenotype. There can be several different wild-type alleles of a specific gene. Also, nucleotide changes in a gene may not affect the phenotype of a subject having two copies of the gene with the nucleotide changes.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region may include, for example, a single nucleotide polymorphism (SNP), the identity of which differs in the different alleles by a single nucleotide at a locus in the polymorphic region of the gene. In another example, a polymorphic region may include a deletion or substitution of one or more nucleotides at a locus in the polymorphic region of the gene.

The expression “amplification of polynucleotides” includes methods such as PCR, ligation amplification (or ligase chain reaction, LCR) and other amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, a PCR procedure is a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e., each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively, the amplified sequence(s) may be cloned prior to sequence analysis. Methods for direct cloning and sequence analysis of enzymatically amplified genomic segments are known in the art.

The term “encode,” as it is applied to polynucleotides, refers to a polynucleotide which is said to “encode” a polypeptide. The polynucleotide is transcribed to produce mRNA, which is then translated into the polypeptide and/or a fragment thereof by cell machinery. An antisense strand is the complement of such a polynucleotide, and the encoding sequence can be deduced therefrom.

As used herein, the term “gene” or “recombinant gene” refers to a nucleic acid molecule comprising an open reading frame and including at least one exon and optionally an intron sequence. The term “intron” refers to a DNA sequence present in a given gene which is spliced out during mRNA maturation.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A “related” or “homologous” sequence shares identity with a comparative sequence, such as 100%, at least 99%, at least 95%, at least 90%, at least 80%, at least 70%, at least 60%, at least 50%, at least 40%, at least 30%, at least 20%, or at least 10%. An “unrelated” or “non-homologous” sequence shares less identity with a comparative sequence, such as less than 95%, less than 90%, less than 80%, less than 70%, less than 60%, less than 50%, less than 40%, less than 30%, less than 20%, or less than 10%.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

The term “isolated” as used herein with respect to nucleic acids, such as DNA or RNA, refers to molecules separated from other DNAs or RNAs, respectively, which are present in a natural source of a macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

As used herein, the term “nucleic acid” refers to polynucleotides such as deoxyribonucleic acid (DNA), and, where appropriate, ribonucleic acid (RNA). The term “nucleic acid” should also be understood to include, as equivalents, derivatives, variants and analogs of either RNA or DNA made from nucleotide analogs, and, as applicable to the embodiment being described, single (sense or antisense) and double-stranded polynucleotides.

Deoxyribonucleotides include deoxyadenosine, deoxycytidine, deoxyguanosine, and deoxythymidine. For purposes of clarity, when referring herein to a nucleotide of a nucleic acid, which can be DNA or RNA, the terms “adenosine,” “cytidine,” “guanosine,” and “thymidine” are used. It is understood that if the nucleic acid is RNA, it includes nucleotide(s) having a uracil base that is uridine.

The terms “oligonucleotide” or “polynucleotide,” or “portion,” or “segment” thereof refer to a stretch of polynucleotide residues which may be long enough to use in PCR or various hybridization procedures to identify or amplify identical or related parts of mRNA or DNA molecules. The polynucleotide compositions described herein may include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications can include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). This may also include synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The phrase “genetic profile” is used interchangeably with “genotype information” and refers to part or all of an identified genotype of a subject and may include one or more polymorphisms in one or more genes of interest. A genetic profile may not be limited to specific genes and polymorphisms described herein, and can include any number of other polymorphisms, gene expression levels, polypeptide sequences, or other genetic markers that are associated with a subject or patient.

The term “patient” refers to an individual waiting for or under medical care and treatment, such as a treatment for medical condition. While the disclosed methods are designed for human patients, such methods are applicable to any suitable individual, which includes, but is not limited to, a mammal, such as a mouse, rat, rabbit, hamster, guinea pig, cat, dog, goat, cow, horse, pig, and simian. Human patients include male and female patients of any ethnicity. The term “treating” as used herein is intended to encompass curing as well as ameliorating at least one symptom of a condition or disease.

The nucleic acid codes utilized herein include: A for Adenine, C for Cytosine, G for Guanine, T for Thymine, and U for Uracil.

As used herein, the terms “drug,” “medication,” and “therapeutic compound” or “compound” are used interchangeably and refer to any chemical entity, pharmaceutical, drug, biological, and the like that can be used to treat or prevent a disease, illness, condition, or disorder of bodily function. A drug may comprise both known and potentially therapeutic compounds. A drug may be determined to be therapeutic by screening using the screening known to those having ordinary skill in the art. A “known therapeutic compound” or “medication” refers to a therapeutic compound that has been shown (e.g., through animal trials or prior experience with administration to humans) to be effective in such treatment. Examples of drugs include, but are not limited to peptides, polypeptides, synthetic organic molecules, naturally occurring organic molecules, nucleic acid molecules, and combinations thereof.

The biological basis for an outcome in a specific patient following a treatment with an opioid medication is subject to, inter alia, the patient's genetic predisposition to opioid response. It has been determined that select polymorphisms of a patient, including single nucleotide permutations, haplotypes and phenotypes may be utilized to generate genotype information. The genotype information may be utilized to generate prognostic information. The prognostic information may be utilized in determining treatment options for the patient. The prognostic information is then based on the patient's genetic predisposition to opioid response. The prognostic information may also be utilized in determining an expected outcome of a treatment of an individual, such as a treatment with an opioid medication.

When a genetic marker such as a polymorphism is used as a basis for determining a treatment for a patient, as described herein, the genetic marker may be measured before or during treatment. The prognostic information obtained may be used by a clinician in assessing any of the following: (a) a probable or likely suitability of an individual to initially receive opioid medication treatment(s); (b) a probable or likely unsuitability of an individual to initially receive opioid medication treatment(s); (c) a responsiveness of an individual to opioid medication treatment; (d) a probable or likely suitability of an individual to continue to receive opioid medication treatment(s); (e) a probable or likely unsuitability of an individual to continue to receive opioid medication treatment(s); (f) adjusting dosage of an individual receiving opioid medication; and (g) predicting likelihood of clinical benefits of an individual receiving opioid medication. As understood by one of skill in the art, measurement of a genetic marker or polymorphism in a clinical setting can be an indication that this parameter may be used as a basis for initiating, continuing, adjusting and/or ceasing administration of opioid medication treatment, such as described herein.

Select polymorphisms have been indentified that may be utilized for providing prognostic information, according to the principles of the invention. These findings were correlated with various magnitudes of a positive or negative predispositions to opioid response. Accordingly, assaying the genotype at these markers may be utilized to generate prognostic information that may be utilized to predict the expected outcome of treating the patient with an opioid medication based on the expected predisposition of the patient to opioid response. Clinicians prescribing opioid medication and other medications may utilize the prognostic information to improve therapeutic decisions and to avoid treatment failures.

Many of the known human single nucleotide permutations (SNPs) are catalogued by the National Center for Biotechnology Information (NCBI) in the Reference SNP (i.e., “refSNP”) database maintained by NCBI. The Reference SNP database is a polymorphism database (dbSNP) which includes single nucleotide polymorphisms and related polymorphisms, such as deletions and insertions of one or more nucleotides. The database is a public-domain archive maintained by NCBI for a broad collection of simple genetic polymorphisms and can be accessed at http://www.ncbi.nlm.nih.gov/snp.

A number of patients have experienced health problems associated with the lack of efficacy of certain opioids in specific individuals. Numerous investigations have demonstrated that this phenomenon may be, in part, attributed to the broad variability in individual response profiles and to genetic polymorphisms in candidate genes involved in immunological and inflammatory signaling pathways. Using these polymorphisms to identify patients at risk of poor opioid response would play an important role in modulating opioid response.

DNA polymorphisms have been identified that may be utilized according to the principles of the invention include SNPs and haplotypes associated with genetic markers in several genes. The genes include the respective genes encoding opioid receptor, mu 1 (OPRM1), solute carrier family 6 (neurotransmitter transporter) member 3 (SLC6A3), Catechol O-Methyltransferase (COMT), the ATP-binding cassette sub-family B member 1 (ABCB1), solute carrier family 22 (SLC22A1 OCT A>C), 5-Hydroxytryptamine (Serotonin) Receptor 2A, G Protein-Coupled (HT2RA), (GABA)-A Receptor, gamma 2 subunit (GABRG2), solute carrier family 6 (neurotransmitter transporter) member 4 (SLC6A4), Cytochrome P450 3A4 (CYP3A4), Cytochrome P450 3A5 (CYP3A5), and Cytochrome P450 2D6 (CYP2D6).

SNP diploid polymorphisms associated with these genes are described throughout the tables below. CYP haplotype polymorphisms for CYP3A4 and CYP2D6 are identified in Table 3 below while CYP haplotype polymorphisms for CYP3A5 are described in Table 10 below. All these CYP haplotype polymorphisms are further described in U.S. Utility Application No. TBD based on Attorney Docket No. P7916US02, entitled “System and Method for Processing Genotype Information Relating to Drug Metabolism” by Brian Meshkin filed on TBD, which has been incorporated herein by reference in its entirety.

The panel of genetic markers describe above can be used to predict several factors associated with an individual's response to opioid medications. An opioid response can be assessed using the polymorphisms found in these genes, as well as by characterizing the patient's metabolic profile, as genetic polymorphisms in metabolizing enzymes can be regarded as one of the principal causes of inter-individual variation in response to medications and in development of adverse reactions.

The DNA polymorphisms which have been identified as active for predicting a genetic predisposition to opioid response are SNP diploid polymorphisms. In the identified SNP diploid polymorphisms, the predisposition to opioid response varies depending upon the active allele of a SNP in a chromosome of a gene as well as the zygosity of the SNP diploid at the locus of the SNP on the chromosome.

For example, a method provided by the invention is a diagnostic method for determining the Opioid Response associated with a patient which method is not practised on the patient's body, i.e. is an ex vivo diagnostic method. The method may involve determining patient information which may be obtained by assaying a sample of genetic material associated with the patient. The method does not involve obtaining the sample from the patient's body. The invention also provides uses of the systems and methods, for example of the diagnostic assays, for determining the Opioid Response risk associated with a patient.

Hydrocodone

For hydrocodone, the SNP diploid polymorphisms identified as having a an identified predisposition to select opioid responses are listed below. In particular, Table 1 identifies the SNP diploid polymorphs associated with hydrocodone response.

TABLE 1*
Identification of SNP Diploid Polymorphisms-Hydrocodone

| | | SNP | | | |
|---|---|---|---|---|---|
| | | Diploid | | | |
| No. | rs# | ID** | Zygosity | DNA Context Sequence for Active SNP(s)*** | SEQ ID |
| 1 | rs1799971 | OPRM1- | homozygous | GGTCAACTTGTCCCACTTAGATGGC[A]ACCTGTCCGACCCATGCGG | SEQ ID No: 1 |
| | | ANC | | TCCGAAC | |
| 2 | rs1799971 | OPRM1- | heterozygous | GGTCAACTTGTCCCACTTAGATGGC[A/G]ACCTGTCCGACCCATGC | SEQ ID No: 2 |
| | | HET | | GGTCCGAAC | |
| 3 | rs1799971 | OPRM1- | homozygous | GGTCAACTTGTCCCACTTAGATGGC[G]ACCTGTCCGACCCATGCGG | SEQ ID No: 3 |
| | | NONA | | TCCGAAC | |
| 4 | rs27072 | SLC6A3- | homozygous | AGTGCCCCTGGGGCAGCCTCAGAGC[C]GGGAGCAGGGAGCAGGGAG | SEQ ID No: 4 |
| | | ANC | | GGAGGG | |
| 5 | rs27072 | SLC6A3- | heterozygous | AGTGCCCCTGGGGCAGCCTCAGAGC[C/T]GGGAGCAGGGAGCAGGG | SEQ ID No: 5 |
| | | HET | | AGGGAGGG | |
| 6 | rs27072 | SLC6A3- | homozygous | AGTGCCCCTGGGGCAGCCTCAGAGC[T]GGGAGCAGGGAGCAGGGAG | SEQ ID No: 6 |
| | | NONA | | GGAGGG | |

*Unless otherwise indicated, the context sequences are in FASTA format, as presented by NCBI within the rs cluster
report identified by “rs#” in the NCBI SNP reference database accessible at http://www.ncbi.nlm.nih.gov/snp.
**The naming conventions for the SNP Diploid Polymorphisms indicate the diploid is either -ANC (homozygous for the
ancestral SNP), -HET (heterozygous as including one ancestral and one non-ancestral SNP in the diploid), or -NONA
(homozygous for the non-ancestral SNP).
***Brackets (i.e. “[. . .]”) appear within each context sequence to indicate the location (i.e., the “polymorphism
marker” or “marker”) of the polymorphic region in the context sequence.

In Table 1, the active polymorphisms are the various diploid pair of alleles associated with “SNP markers” called “rs numbers” in the ref SNP database. Different diploid pairs for each allele have varying activities for generating prognostic information about hydrocodone response. A SNP marker in dbSNP references a SNP cluster report identification number (i.e., the “rs number”) in the ref SNP database. The context sequences shown in Table 1 include the allelic variant(s) and the zygosity of the diploid pair identified as active for providing prognostic information according to the principles of the invention. The context sequences include the active polymorphism SNP located in the relevant region of the gene. The context sequences also include a number of nucleotide bases flanking the active polymorphism SNP in the relevant region of the gene. In the context sequences shown in Table 1, the polymorphic SNP location is shown in brackets within the context sequence for identification purposes. Table 1 also show the rs cluster report number (i.e., the “rs number”) associated with the active polymorphism SNP in dbSNP maintained by NCBI.

Studies have been conducted and it has been determined that SNP diploid polymorphisms identified in Table 1 are predictive of a differential predisposition to hydrocodone response associated with a patient having one or more of SNP diploid polymorphisms. Select SNP diploid polymorphisms in Table 1 are associated with a patient having an elevated hydrocodone response (i.e., predisposed to having a higher hydrocodone response).

Hydrocodone “therapy selection” is determined by a score that goes from 0-6: if a patient receives a score of 0-3=“Poor Responder”; and if a patient receives a score of 4-6=“Good Responder.”

The score is determined by summing the following genetic information shown below in Table 2:

TABLE 2
Hydrocodone Genetic Information

| | RS | ANC | ANC | HET | HET | NONA | NONA |
|---|---|---|---|---|---|---|---|
| Gene | Number | Def. | Value | Def. | Value | Def | Value |
| OPRM1 | rs1799971 | AA | 2 | AG | 0 | GG | 0 |
| SLC6A3 | rs27072 | CC | 2 | CT | 2 | TT | 0 |

In addition, select CYP haplotype polymorphisms are identified as associated with hydrocodone risk are listed in Table 3 below. This profile includes an analysis of the enzymes CYP3A4 and CYP2D6, in which the presence of genetic coding variants indicates a risk factor for hydrocodone associated side effects due to a reduction in the enzymes' rate of metabolism. The risk profile combines the evaluation of relevant signalling cascades and metabolizing pathways to provide information regarding hydrocodone-induced risk factors for clinical use and management. Physicians may use this test to determine the likelihood of a patient experiencing an hydrocodone-related adverse event and/or to assist with prescribing hydrocodone at therapeutic doses.

TABLE 3
CYP3A4 and CYP2D6 Haplotype Polymorphisms and Grading
Table 3A CYP3A4 haplotypes @

| CYP3A4 | rs2740574 | rs55785340 | rs4987161 | rs28371759 | rs12721629 | rs35599367 |
|---|---|---|---|---|---|---|
| *1 | T | A | A | A | G | G |
| *1A | T | A | A | A | G | G |
| *1B | C | A | A | A | G | G |
| *1C | T | A | A | A | G | G |
| *1D | T | A | A | A | G | G |
| *1E | T | A | A | A | G | G |
| *1F | T | A | A | A | G | G |
| *1G | T | A | A | A | G | G |
| *1H | T | A | A | A | G | G |
| *1J | T | A | A | A | G | G |
| *1K | T | A | A | A | G | G |
| *1L | T | A | A | A | G | G |
| *1M | T | A | A | A | G | G |
| *1N | T | A | A | A | G | G |
| *1P | T | A | A | A | G | G |
| *1Q | T | A | A | A | G | G |
| *1R | T | A | A | A | G | G |
| *1S | T | A | A | A | G | G |
| *1T | T | A | A | A | G | G |
| *2 | T | G | A | A | G | G |
| *3 | T | A | A | A | G | G |
| *4 | T | A | A | A | G | G |
| *5 | T | A | A | A | G | G |
| *6 | T | A | A | A | G | G |
| *7 | T | A | A | A | G | G |
| *8 | T | A | A | A | G | G |
| *9 | T | A | A | A | G | G |
| *10 | T | A | A | A | G | G |
| *11 | T | A | A | A | G | G |
| *12 | T | A | A | A | A | G |
| *13 | T | A | A | A | G | G |
| *14 | T | A | A | A | G | G |
| *15 | T | A | A | A | G | G |
| *15A | T | A | A | A | G | G |
| *15B | C | A | A | A | G | G |
| *16 | T | A | A | A | G | G |
| *16A | T | A | A | A | G | G |
| *16B | T | A | A | A | G | G |
| *17 | T | A | G | A | G | G |
| *18 | T | A | A | G | G | G |
| *18A | T | A | A | G | G | G |
| *18B | T | A | A | G | G | G |
| *19 | T | A | A | A | G | G |
| *20 | T | A | A | A | G | G |
| *21 | T | A | A | A | G | G |
| *22 | T | A | A | A | G | A |
| *23 | C | A | A | A | G | G |
| *24 | C | A | A | A | G | G |

CYP3A4: Activity Scores

| INCREASED = 1.5 | DECREASED = 0.5 | NORMAL = 1 | NONE = 0 |
|---|---|---|---|
| *18 | *1B | *1A | *22 |
| | *8 | *1 | |
| | *11 | *3 | |
| | *12 | *7 | |
| | *13 | *9 | |
| | *16 | *10 | |
| | *17 | *19 | |
| | *2 | | |

CYP3A4: Grading based on Activity Scores

| | |
|---|---|
| D | 0 = null/null |
| C | 0.5 = null/reduced |
| C | 1 = reduced/reduced OR normal/null |
| B | 1.5 = reduced/normal |
| B | 2-2.5 = normal/normal or normal/increased |
| A | >2.5 = increased/increased |

TABLE 3B
CYP2D6 haplotypes @

| CYP2D6 | rs1080985 [symbol] | rs35742686 [symbol] | rs3892097 [symbol] | rs5030655 [symbol] | rs5030867 [symbol] | rs5030865 [symbol] | rs1065852 [symbol] | rs72549357 [symbol] |
|---|---|---|---|---|---|---|---|---|
| *1 | G | T | C | A | T | C | G | del |
| *71 | C | T | C | A | T | C | G | del |
| *15 | G | T | C | A | T | C | G | insA |
| Report *39 (default). | G | T | C | A | T | C | G | del |
| *70 | G | T | C | A | T | C | G | del |
| *7 | G | T | C | A | G | C | G | del |
| *34 | G | T | C | A | T | C | G | del |
| *63 | C | T | C | A | T | C | G | del |
| Report *2 (default). | G | T | C | A | T | C | G | del |
| *20 | G | T | C | A | T | C | G | del |
| *14 | G | T | C | A | T | T | G | del |
| *11 | G | T | C | A | T | C | G | del |
| Report “*2A or *21”. | C | T | C | A | T | C | G | del |
| *29 | G | T | C | A | T | C | G | del |
| *91 | G | T | C | A | T | C | G | del |
| *41 | G | T | C | A | T | C | G | del |
| *9 | G | T | C | A | T | C | G | del |
| Report *3 (default). | G | delT | C | A | T | C | G | del |
| *4M | G | T | T | A | T | C | G | del |
| *6 | G | T | C | delA | T | C | G | del |
| *6C | G | T | C | delA | T | C | G | del |
| Report *17 (default). | G | T | C | A | T | C | G | del |
| *12 | G | T | C | A | T | C | G | del |
| *68A | G | T | C | A | T | C | A | del |
| Report “*10 or [symbol] 36”. | G | T | C | A | T | C | A | del |
| *65 | G | T | C | A | T | C | A | del |
| *14A | G | T | C | A | T | T | A | del |
| *69 | G | T | C | A | T | C | A | del |
| Report *4 (default). | G | T | T | A | T | C | A | del |
| Report *4 (default). | G | T | T | A | T | C | A | del |
| *4K | G | T | T | A | T | C | A | del |
| *64 | G | T | C | A | T | C | A | del |
| Report *35 (default). | C | T | C | A | T | C | G | del |

| rs28371706 [symbol] | rs59421388 [symbol] | rs769258 [symbol] | rs16947 [symbol] | rs1135840 [symbol] | rs28371725 [symbol] | rs5030656 [symbol] | rs201377835 [symbol] | rs5030862 [symbol] | rs7254935 [symbol] |
|---|---|---|---|---|---|---|---|---|---|
| G | C | C | G | C | C | CTT | C | C | del |
| G | C | C | G | C | C | CTT | C | C | del |
| G | C | C | G | C | C | CTT | C | C | del |
| G | C | C | G | G | C | CTT | C | C | del |
| G | T | C | G | G | C | CTT | C | C | del |
| G | C | C | G | C | C | CTT | C | C | del |
| G | C | C | A | C | C | CTT | C | C | del |
| G | C | C | A | C | C | CTT | C | C | del |
| G | C | C | A | G | C | CTT | C | C | del |
| G | C | C | A | G | C | CTT | C | C | insC |
| G | C | C | A | G | C | CTT | C | C | del |
| G | C | C | A | G | C | CTT | G | C | del |
| G | C | C | A | G | C | CTT | C | C | del |
| G | T | C | A | G | C | CTT | C | C | del |
| G | C | C | A | C | T | CTT | C | C | del |
| G | C | C | A | G | T | CTT | C | C | del |
| G | C | C | G | C | C | delCTT | C | C | del |
| G | C | C | G | C | C | CTT | C | C | del |
| G | C | C | G | C | C | CTT | C | C | del |
| G | C | C | G | C | C | CTT | C | C | del |
| G | C | C | G | G | C | CTT | C | C | del |
| A | C | C | A | G | C | CTT | C | C | del |
| G | C | C | A | G | C | CTT | C | T | del |
| G | C | C | G | C | C | CTT | C | C | del |
| G | C | C | G | G | C | CTT | C | C | del |
| G | C | C | A | G | C | CTT | C | C | del |
| G | C | C | A | G | C | CTT | C | C | del |
| G | C | C | A | G | T | CTT | C | C | del |
| G | C | C | G | C | C | CTT | C | C | del |
| G | C | C | G | G | C | CTT | C | C | del |
| G | C | C | A | G | C | CTT | C | C | del |
| A | C | C | G | G | C | CTT | C | C | del |
| G | C | T | A | G | C | CTT | C | C | del |

CYP2D6: Star Allele Scoring

| increased = 1.5 | reduced = 0.5 | null = 0 | normal = 1 |
|---|---|---|---|
| *1XN | *9 | *3 | *1 |
| *2XN | *10 | *4A-D | *2A |
| *35X2 | *10X2 | *4K | *33 |
| | *17 | *4X2 | *35 |
| | *29 | *5 | *27 |
| | *41 | *6 | *33 |
| | *69 | *7 | *35 |
| | *91 | *8 | *45 |
| | *49 | *11 | *46 |
| | *50 | *12 | *39 |
| | *54 | *13 | *48 |
| | *55 | *14 | *53 |
| | *59 | *15 | *2 |
| | *69 | *18 | *71 |
| | *72 | *19 | *70 |
| | *146 | *20 | *34 |
| | | *21A-B | *63 |
| | | *31 | *65 |
| | | *38 | *64 |
| | | *40 | |
| | | *42 | |
| | | *44 | |
| | | *4 | |
| | | *16 | |
| | | *36 | |
| | | *47 | |
| | | *51 | |
| | | *56 | |
| | | *57 | |
| | | *62 | |
| | | *4M | |
| | | *6C | |
| | | *14A | |
| | | *3A | |
| | | *6A | |
| | | *66 | |
| | | *68A | |

| | CYP2D6: Grading | | |
|---|---|---|---|
| Grade | Activity score | Functional Allele diplotype | [symbol] |
| D | 0 | null/null | [symbol] |
| D | 0.5 | null/reduced | [symbol] |
| D | 1 | reduced/reduced OR normal/nu [symbol] | [symbol] |
| C | 1.5 | normal/reduced function | [symbol] |
| B | 2 | normal/normal | [symbol] |
| A | >2 | duplicates of functional alleles | [symbol] |

[symbol]

indicates data missing or illegible when filed

@ Unless otherwise indicated, context sequences in FASTA format, are presented by NCBI within the rs cluster report identified by “rs#” associated with each rs number in Tables 3A and 3B above in the NCBI SNP reference database accessible at http://www.ncbi.nlm.nih.gov/snp, and which is incorporated by reference herein for each recited SNP rs number in the Table(s) above.

For CYP haplotypes, with respect to hydrocodone risk assessment, an exemplary algorithm for determining hydrocodone mediated side effect risk is shown below. Each category is scored separately as shown in the charts below, but all are based on the following scoring system. As would be known by one of ordinary skill in the art, there are four general categories of CYP star alleles (i.e., CYP haplotypes): normal function, reduced function, null function and increased function. The nomenclature is reported by, for example, Robarge et al., “The Star-Allele Nomenclature: Retooling for Translational Genomics” Nature, v. 82, no. 3, September 2007, pp. 244-248, which is incorporated by reference herein.

A large number of star alleles have been reported for each cytochrome. Among these are normal functioning CYP star alleles, CYP star alleles with some function that is a reduced function, CYP star alleles with null (or non-functional) alleles, and CYP star alleles with increased functionality. These alleles convey a wide range of enzyme activity, from no activity to ultrarapid metabolism of substrates/medications.

For CYP haplotypes shown in Table 3 above, the categorization of the CYP3A4 and CYP2D6 haplotypes which are associated with an individual are graded as an A, B, C, or D. The grade applied to the DNA information associated with the individual is obtained by determining which two star allele(s) the individual has by identifying the CYP3A4 and CYP2D6 haplotypes, assigning a score for the two alleles present in the individual for each gene and then assigning a grade for each gene in the individual based on their added score. For example, an individual is determined to have the following two CYP3A4 star alleles: CYP3A4*1 and CYP3A4*8. The allele score for CYP3A4*1=1.0 and the allele score for CYP3A4*8=0.5. These are summed to provide an activity score for the individual of 1.5. Thus the undividual is assigned a grade of “B” according to the Activity Scoring for CYP3A4 in Table 3. For hydrocodone prognostic information, this grading is performed for both CYP3A4 and CYP2D6 haplotypes in the individual, according to Table 3.

The haplotypes for the above mentioned CYP star alleles are also described in U.S. Utility Application No. TBD based on Attorney Docket No. P7916PC01 entitled “System and Method for Processing Genotype Information Relating to Drug Metabolism” by Brian Meshkin filed on Apr. 28, 2016, which has been incorporated herein by reference in its entirety.

Using the Table 3 above to score the raw results; then applying these scores to the Tables below to arrive at the interpretations reported on the tests.

Hydrocodone is metabolized by both CYP3A4 and CYP2D6, and the dosing recommendations for this test are determined as shown in Table 5 below.

TABLE 5
Hydrocodone Dosing Recommendations
CYP3A4

| | | A | B | C | D |
|---|---|---|---|---|---|
| CYP2D6 | A | This patient | This patient is at | This patient is at | This patient is at |
| | | may experience | risk of | risk of | risk of |
| | | insufficient pain | experiencing an | experiencing an | experiencing an |
| | | relief with | adverse drug | adverse drug | adverse drug |
| | | average doses of | event with | event with | event with |
| | | Hydrocodone. | Hydrocodone. | Hydrocodone. | Hydrocodone. |
| | | Monitor the | Consider lowering | Consider | Consider |
| | | patient's | the dose of the | lowering the | lowering the |
| | | analgesic | medication. | dose of the | dose of the |
| | | response and | | medication. | medication. |
| | | consider | | | |
| | | prescribing an | | | |
| | | increased dose | | | |
| | | if necessary. | | | |
| | B | This patient is | This patient is | This patient is | This patient is at |
| | | predicted to be a | predicted to be a | predicted to be a | risk of |
| | | normal | normal | normal | experiencing an |
| | | metabolizer of | metabolizer of | metabolizer of | adverse drug |
| | | Hydrocodone. | Hydrocodone. | Hydrocodone. | event with |
| | | Prescribe at | Prescribe at | Prescribe at | Hydrocodone. |
| | | standard label | standard label | standard label | Consider |
| | | recommendations. | recommendations. | recommendations. | lowering the |
| | | no change | no change | no change | dose of the |
| | | | | | medication. |
| | C | This patient | This patient may | This patient may | This patient may |
| | | may experience | experience | experience | experience |
| | | insufficient pain | insufficient pain | insufficient pain | insufficient pain |
| | | relief with | relief with | relief with | relief with |
| | | average doses of | average doses of | average doses of | average doses of |
| | | Hydrocodone. | Hydrocodone. | Hydrocodone | Hydrocodone |
| | | Monitor the | Monitor the | and/or is at | and/or is at |
| | | patient's | patient's analgesic | increased risk of | increased risk of |
| | | analgesic | response and | experiencing an | experiencing an |
| | | response and | consider | adverse drug | adverse drug |
| | | consider | prescribing an | event. Consider | event. Consider |
| | | prescribing an | increased dose if | choosing an | choosing an |
| | | increased dose | necessary. | alternative | alternative |
| | | if necessary. | | medication. | medication. |
| | D | This patient | This patient may | This patient may | This patient may |
| | | may experience | experience | experience | experience |
| | | insufficient pain | insufficient pain | insufficient pain | insufficient pain |
| | | relief with | relief with | relief with | relief with |
| | | average doses of | average doses of | average doses of | average doses of |
| | | Hydrocodone. | Hydrocodone. | Hydrocodone | Hydrocodone |
| | | Monitor the | Monitor the | and/or is at | and/or is at |
| | | patient's | patient's analgesic | increased risk of | increased risk of |
| | | analgesic | response and | experiencing an | experiencing an |
| | | response and | consider | adverse drug | adverse drug |
| | | consider | prescribing an | event. Consider | event. Consider |
| | | prescribing an | increased dose if | choosing an | choosing an |
| | | increased dose | necessary. | alternative | alternative |
| | | if necessary. | | medication. | medication. |
| | B/C/D | This patient is a | This patient is a | This patient is a | This patient is a |
| | | possible | possible | possible | possible |
| | | decreased | decreased | decreased | decreased |
| | | metabolizer of | metabolizer of | metabolizer of | metabolizer of |
| | | Hydrocodone | Hydrocodone into | Hydrocodone | Hydrocodone |
| | | into | Hydromorphone. | into | into |
| | | Hydromorphone | There is a chance | Hydromorphone. | Hydromorphone. |
| | | There is a | that this patient | There is a chance | There is a chance |
| | | chance that this | may experience | that this patient | that this patient |
| | | patient may | insufficient pain | may experience | may experience |
| | | experience | relief. Monitor the | insufficient pain | insufficient pain |
| | | insufficient pain | patient's analgesic | relief. Monitor | relief. Monitor |
| | | relief. Monitor | response and | the patient's | the patient's |
| | | the patient's | increase dose if | analgesic | analgesic |
| | | analgesic | necessary. | response and | response and |
| | | response and | | increase dose if | increase dose if |
| | | increase dose if | | necessary. | necessary. |
| | | necessary. | | | |

As shown above, the investigation of genotypic predisposition for hydrocodone treatment outcomes demonstrated that the C/C-C/T genotype of SLC6A3 are more likely to be associated with good response to hydrocodone treatment compared to the T/T variant. Further, the A/A-A/G genotype of OPRM1 are more likely to be associated with good response to hydrocodone treatment compared to the G/G variant. This test can be used to identify patients who are more likely to be good vs. poor responders to hydrocodone treatment. Alternative pain control measures can be considered based on the results of this test that may lead to better patient outcomes, decreased use of suboptimal medications, and shorter duration of therapy and lower costs.

Pain management remains a challenge for clinicians due to unpredictable patient responses to opioid therapy. Genetic variations in enzymes that produce active metabolites from pro-drugs are well known and can account for some of the clinically observed differences in analgesia and side effects seen in pain patients. For example, some of these variations may result from single nucleotide polymorphisms (SNPs) of the human opioid mu-1 receptor (OPRM1) that modify receptor binding or signal transduction. Determining measurement of serum opioid concentrations and total opioid consumption, provides a more precise method of assessing the effects of OPRM1 on analgesic response. The hepatic cytochrome 2D6 (CYP2D6) is an enzyme responsible for metabolism of approximately 25% of known pharmaceuticals. Hydrocodone is largely dependent on CYP2D6 metabolism for analgesia. Genotyping and quantitation of hydrocodone (and its metabolites) serum concentrations demonstrated the clinical effects of hydrocodone on pain relief in a postoperative study done in 2014. In this study, 156 patient's blood samples reported the CYP2D6 genotype with comparable hydrocodone/hydromorphone levels, which accounted for some of the variability in pain relief seen in postoperative patient's. Pharmacogenetics provides clinicians an opportunity to individualize hydrocodone dosing, while accounting for its conversion to hydromorphone in the body. In another study, CYP2D6 medication coingestion decreased the effectiveness of hydrocodone, and was also found to change the effectiveness of other commonly prescribed drugs. The hydrocodone response profile predicts a patient's genetic response to hydrocodone, and can advise the prescribing physician to any potential adverse drug events.

Hydromorphone

The SNP diploid polymorphisms identified as having a predisposition to response to the opioid hydromorphone are listed below. In particular, Table 4 identifies the SNP diploid polymorphs associated with hydromorphone response.

TABLE 4*
Identification of SNP Diploid Polymorphisms-Morphine

| | | SNP | | | |
|---|---|---|---|---|---|
| No. | rs# | Diploid ID** | Zygosity | DNA Context Sequence for Active SNP(s)*** | SEQ ID |
| 1 | rs1799971 | OPRM1-ANC | homozygous | See above | SEQ ID No: 1 |
| 2 | rs1799971 | OPRM1-HET | heterozygous | See above | SEQ ID No: 2 |
| 3 | rs1799971 | OPRM1-NONA | homozygous | See above | SEQ ID No: 3 |
| 4 | rs4680 | COMT-ANC | homozygous | CCAGCGGATGGTGGATTTCGCTGGC[G]TGAAGGACAAGGT | SEQ ID No: 4 |
| | | | | GTGCATGCCTGA | |
| 5 | rs4680 | COMT-HET | heterozygous | CCAGCGGATGGTGGATTTCGCTGGC[A/G]TGAAGGACAAG | SEQ ID No: 8 |
| | | | | GTGTGCATGCCTGA | |
| 6 | rs4680 | COMT-NONA | homozygous | CCAGCGGATGGTGGATTTCGCTGGC[A]TGAAGGACAAGGT | SEQ ID No: 9 |
| | | | | GTGCATGCCTGA | |

*Unless otherwise indicated, the context sequences are in FASTA format, as presented by NCBI within the rs cluster report
identified by “rs#” in the NCBI SNP reference database accessible at http://www.ncbi.nlm.nih.gov/snp.
**The naming conventions for the SNP Diploid Polymorphisms indicate the diploid is either -ANC (homozygous for the ancestral
SNP), -HET (heterozygous as including one ancestral and one non-ancestral SNP in the diploid), or -NONA (homozygous for the
non-ancestral SNP).
***Brackets (i.e. “[. . .]”) appear within each context sequence to indicate the location (i.e., the “polymorphism marker”
or “marker”) of the polymorphic region in the context sequence.

Studies have been conducted and it has been determined that SNP diploid polymorphisms identified in Table 4 are predictive of a differential predisposition to hydromorphone response associated with a patient having one or more of SNP diploid polymorphisms. Select SNP diploid polymorphisms in Table 4 are associated with a patient having an elevated hydromorphone response (i.e., predisposed to having a higher hydromorphone response).

Hydromorphone “therapy selection” is determined by a score that goes from 0-4: if a patient receives a score of 0-2=“Poor Responder”; and if a patient receives a score of 3-4=“Good Responder.”

The score is determined by summing the following genetic information shown below in Table 5:

TABLE 5
Hydromorphone Genetic Information

| | RS | ANC | ANC | HET | HET | NON-A | NON-A |
|---|---|---|---|---|---|---|---|
| Gene | Number | Def. | Value | Def. | Value | Def | Value |
| OPRM1 | rs1799971 | AA | 2 | AG | 0 | GG | 0 |
| COMT | rs4680 | GG | 0 | GA | 2 | AA | 2 |

As shown in Table 5, for COMT (rs4680): A/A-A/G is more associated with a good response while G/G is more associated with a poor response to hydromorphone. This test can be used to identify patients who are more likely to be good vs. poor responders to hydromorphone.

Genetic variations in enzymes that produce active metabolites from pro-drugs can account for some of the clinically observed differences in analgesia and side effects seen in pain patients. Pain management remains a challenge for clinicians due to unpredictable patient responses to opioid therapy. Studies provide information regarding distribution of important single-nucleotide polymorphisms (SNPs) and evaluate their associations with clinical response to intravenous hydromorphone. Hydromorphone is generated at substantially different rates, dependent on CYP2D6 genotype. Variability of a patient's response to hydromorphone may be due to SNPs of the human opioid mu-1 receptor (OPRM1) that modify receptor binding or signal transduction. The inability to metabolize hydrocodone to hydromorphone, as seen in poor metabolizers, should alert the clinician to consider alternative medications for managing pain. Measurement of serum opioid concentrations is recommended when assessing the role of OPRM1 variants in pain relief. The response profile predicts a patient's genetic response to hydromorphone, and can advise the prescribing physician to any potential adverse drug events and assist the physician with properly prescribing hydromorphone at optimal doses for each patient's individual needs.

Morphine

Morphine is a well-known opioid. The SNP diploid polymorphisms identified as having a predisposition to response to morphine are listed below. In particular, Table 6 identifies the SNP diploid polymorphs associated with morphine response.

TABLE 6*
Identification of SNP Diploid Polymorphisms-Morphine

| | | SNP | | | |
|---|---|---|---|---|---|
| No. | rs# | Diploid ID** | Zygosity | DNA Context Sequence for Active SNP(s)*** | SEQ ID |
| 1 | rs1799971 | OPRM1-ANC | homozygous | See above | SEQ ID No: 1 |
| 2 | rs1799971 | OPRM1-HET | heterozygous | See above | SEQ ID No: 2 |
| 3 | rs1799971 | OPRM1-NONA | homozygous | See above | SEQ ID No: 3 |
| 4 | rs1045642 | ABCB1(C3435T)- | homozygous | GCCGGGTGGTGTCACAGGAAGAGAT[C]GTGAGGGCAGCAA | SEQ ID No: 10 |
| | | ANC | | AGGAGGCCAACA | |
| 5 | rs1045642 | ABCB1(C3435T)- | heterozygous | GCCGGGTGGTGTCACAGGAAGAGAT[A/C/T]GTGAGGGCA | SEQ ID No: 11 |
| | | HET | | GCAAAGGAGGCCAACA | |
| 6 | rs1045642 | ABCB1(C3435T)- | homozygous | GCCGGGTGGTGTCACAGGAAGAGAT[T]GTGAGGGCAGCAA | SEQ ID No: 12 |
| | | NONA | | AGGAGGCCAACA | |
| 7 | rs1128503 | ABCB1(C1236T)- | homozygous | ACTCGTCCTGGTAGATCTTGAAGGG[C]CTGAACCTGAAGG | SEQ ID No: 13 |
| | | ANC | | TGCAGAGTGGGC | |
| 8 | rs1128503 | ABCB1(C1236T)- | heterozygous | ACTCGTCCTGGTAGATCTTGAAGGG[C/T]CTGAACCTGAA | SEQ ID No: 14 |
| | | HET | | GGTGCAGAGTGGGC | |
| 9 | rs1128503 | ABCB1(C1236T)- | homozygous | ACTCGTCCTGGTAGATCTTGAAGGG[T]CTGAACCTGAAGG | SEQ ID No: 15 |
| | | NONA | | TGCAGAGTGGGC | |
| 10 | rs2032582 | ABCB1(G2677A/T)- | homozygous | GAAAGATAAGAAAGAACTAGAAGGT[G]CTGGGAAGGTGAG | SEQ ID No: 16 |
| | | ANC | | TCAAACTAAATA | |
| 11 | rs2032582 | ABCB1(G2677A/T)- | heterozygous | GAAAGATAAGAAAGAACTAGAAGGT[A/G/T]CTGGGAAGG | SEQ ID No: 17 |
| | | HET | | TGAGTCAAACTAAATA | |
| 12 | rs2032582 | ABCB1(G2677A/T)- | homozygous | GAAAGATAAGAAAGAACTAGAAGGT[T]CTGGGAAGGTGAG | SEQ ID No: 18 |
| | | NONA | | TCAAACTAAATA | |
| 13 | rs622342 | SLC22A1_OCT | homozygous | TTCTTCAAATTTGATGAAAACTTC[A]AATACATAGATCTA | SEQ ID No: 19 |
| | | A > C-ANC | | ACAATCTAAT | |
| 14 | rs622342 | SLC22A1_OCT | heterozygous | TTCTTCAAATTTGATGAAAACTTC[A/C]AATACATAGATC | SEQ ID No: 20 |
| | | A > C-HET | | TAACAATCTAAT | |
| 15 | rs622342 | SLC22A1_OCT | homozygous | TTCTTCAAATTTGATGAAAACTTC[C]AATACATAGATCTA | SEQ ID No: 21 |
| | | A > C-NONA | | ACAATCTAAT | |

*Unless otherwise indicated, the context sequences are in FASTA format, as presented by NCBI within the rs cluster report
identified by “rs#” in the NCBI SNP reference database accessible at http://www.ncbi.nlm.nih.gov/snp.
**The naming conventions for the SNP Diploid Polymorphisms indicate the diploid is either -ANC (homozygous for the ancestral
SNP), -HET (heterozygous as including one ancestral and one non-ancestral SNP in the diploid), or -NONA (homozygous for the
non-ancestral SNP).
***Brackets (i.e. “[. . .]”) appear within each context sequence to indicate the location (i.e., the “polymorphism marker”
or “marker”) of the polymorphic region in the context sequence.

Studies have been conducted and it has been determined that SNP diploid polymorphisms identified in Table 6 are predictive of a differential predisposition to morphine response associated with a patient having one or more of SNP diploid polymorphisms. Select SNP diploid polymorphisms in Table 6 are associated with a patient having an elevated morphine response (i.e., predisposed to having a higher morphine response).

Morphine “therapy selection” is determined by a score that goes from 0-10: if a patient receives a score of 0-5=“Poor Responder”; and if a patient receives a score of 6-10=“Good Responder.”

The score is determined by summing the following genetic information shown below in Table 7:

TABLE 7
Morphine Genetic Information

| | Other Gene | RS | ANC | ANC | HET | HET | NONA | NONA |
|---|---|---|---|---|---|---|---|---|
| Gene | Names | Number | Def. | Value | Def. | Value | Def | Value |
| ABCB1 | C3435T | rs1045642 | CC | 0 | CT | 0 | TT | 2 |
| ABCB1 | C1236T | rs1128503 | CC | 0 | CT | 0 | TT | 2 |
| ABCB1 | G2677A/T | rs2032582 | GG | 2 | GT | 1 | TT | 1 |
| ABCB1 | G2677A/T | rs2032582 | AT | 0 | GT | 0 | TT | 0 |
| OPRM1 | C118 A_G | rs1799971 | AA | 2 | AG | 0 | GG | 0 |
| SLC22A1_OCT | 1386- | rs622342 | AA | 0 | AC | 2 | CC | 2 |
| A > C | 2964C > A | | | | | | | |

As shown in Table 7, for OPRM1(rs 1799971): A/A is more associated with good response; and A/G-G/G is more associated with poor response to morphine.

The gene variants ABCB1, OPRM1, and OCT1 have been associated with response to opioid analgesia. In studies many polymorphisms have been found to contribute to inter-individual variability regarding pain sensitivity, opioid usage, tolerance and dependence and incidence of adverse effects in patients with chronic pain. Although a number of studies have investigated these associations, a consensus has not yet been reached. The OPRM1 A118G polymorphism was associated with inter-individual variability in response to opioids, such as morphine. OCT1 plays an important role in the hepatocellular uptake of morphine. Polymorphisms of the ATP-binding cassette sub-family B member-1 (ABCB1) gene that codes for P-glycoprotein was found to influence the efflux of morphine from the central nervous system affecting its analgesic action. The response profile predicts a patient's genetic response to morphine medications, and can advise the prescribing physician to any potential adverse drug events and assist the physician with properly prescribing morphine at optimal doses for each patient's individual needs.

Oxycodone

Oxycodone is a semi-synthetic opioid with a mu-receptor (OPRM1) agonist-mediated effect in many pain conditions. Genetic variations of the OPRM1 gene that encodes the mu-opioid receptor is of great interest for understanding pain management. Oxycodone is metabolized by CYP2D6 and CYP3A. CYP interactions were found to alter the efficacy of many prescribed medications, including oxycodone. Studies have shown the effect of CYP2D6, CYP3A5, ABCB1, and OPRM1 polymorphisms in predicting central nervous system depression after oxycodone use. These studies also show that CYP2D6 drug-drug interactions appear to change effectiveness of commonly prescribed drugs. Drug-drug interaction should be considered prior to prescribing CYP2D6 drugs, such as oxycodone. Researchers have found an association between the single-nucleotide polymorphisms and the analgesic effect of intravenous oxycodone in pain management. OPRM1 is a significant factor for determining oxycodone requirement for many pain patients. Marked differences were found in the antinociceptive properties of oxycodone and morphine. The response profile predicts a patient's genetic response to oxycodone medications, and can advise the prescribing physician to any potential adverse drug events, and can assist the physician with properly prescribing oxycodone at optimal doses for each patient's individual needs.

The SNP diploid polymorphisms identified as having a predisposition to response to the opioid oxycodone are listed below. In particular, Table 8 identifies the SNP diploid polymorphs associated with oxycodone response.

TABLE 8*
Identification of SNP Diploid Polymorphisms-Oxycodone

| | | SNP | | | |
|---|---|---|---|---|---|
| No. | rs# | Diploid ID** | Zygosity | DNA Context Sequence for Active SNP(s)*** | SEQ ID |
| 1 | rs1045642 | ABCB1(C3435T)- | homozygous | See above | SEQ ID No: 10 |
| | | ANC | | | |
| 2 | rs1045642 | ABCB1(C3435T)- | heterozygous | See above | SEQ ID No: 11 |
| | | HET | | | |
| 3 | rs1045642 | ABCB1(C3435T)- | homozygous | See above | SEQ ID No: 12 |
| | | NONA | | | |
| 4 | rs2032582 | ABCB1(G2677A/T)- | homozygous | See above | SEQ ID No: 16 |
| | | ANC | | | |
| 5 | rs2032582 | ABCB1(G2677A/T)- | heterozygous | See above | SEQ ID No: 17 |
| | | HET | | | |
| 6 | rs2032582 | ABCB1(G2677A/T)- | homozygous | See above | SEQ ID No: 18 |
| | | NONA | | | |
| 7 | rs1799971 | OPRM1-ANC | homozygous | See above | SEQ ID No: 1 |
| 8 | rs1799971 | OPRM1-HET | heterozygous | See above | SEQ ID No: 2 |
| 9 | rs1799971 | OPRM1-NONA | homozygous | See above | SEQ ID No: 3 |
| 10 | rs7997012 | HT2RA-ANC | homozygous | TGCCATTATCTTCAAAGACTTAATT[G]ACAATATTTGTCA | SEQ ID No: 22 |
| | | | | CTTGCCTATGCA | |
| 11 | rs7997012 | HT2RA-HET | heterozygous | TGCCATTATCTTCAAAGACTTAATT[A/G]ACAATATTTGT | SEQ ID No: 23 |
| | | | | CACTTGCCTATGCA | |
| 12 | rs7997012 | HT2RA-NONA | homozygous | TGCCATTATCTTCAAAGACTTAATT[A]ACAATATTTGTCA | SEQ ID No: 24 |
| | | | | CTTGCCTATGCA | |
| 13 | rs4680 | COMT-ANC | homozygous | See above | SEQ ID No: 7 |
| 14 | rs4680 | COMT-HET | heterozygous | See above | SEQ ID No: 8 |
| 15 | rs4680 | COMT-NONA | homozygous | See above | SEQ ID No: 9 |
| 16 | rs211014 | GABRG2-ANC | homozygous | GCAGGCTAAGGCTCAGCAGTTTGGG[C]TCCAAGATGAAAA | SEQ ID No: 25 |
| | | | | CAGCATGTATGA | |
| 17 | rs211014 | GABRG2-HET | heterozygous | GCAGGCTAAGGCTCAGCAGTTTGGG[C/A]TCCAAGATGAA | SEQ ID No: 26 |
| | | | | AACAGCATGTATGA | |
| 18 | rs211014 | GABRG2-NONA | homozygous | GCAGGCTAAGGCTCAGCAGTTTGGG[A]TCCAAGATGAAAA | SEQ ID No: 27 |
| | | | | CAGCATGTATGA | |

*Unless otherwise indicated, the context sequences are in FASTA format, as presented by NCBI within the rs cluster report
identified by “rs#” in the NCBI SNP reference database accessible at http://www.ncbi.nlm.nih.gov/snp.
**The naming conventions for the SNP Diploid Polymorphisms indicate the diploid is either -ANC (homozygous for the ancestral
SNP), -HET (heterozygous as including one ancestral and one non-ancestral SNP in the diploid), or -NONA (homozygous for the
non-ancestral SNP).
***Brackets (i.e. “[. . .]”) appear within each context sequence to indicate the location (i.e., the “polymorphism marker”
or “marker”) of the polymorphic region in the context sequence.

Studies have been conducted and it has been determined that SNP diploid polymorphisms identified in Table 8 are predictive of a differential predisposition to oxycodone response associated with a patient having one or more of SNP diploid polymorphisms. Select SNP diploid polymorphisms in Table 9 are associated with a patient having an elevated oxycodone response (i.e., predisposed to having a higher oxycodone response).

Oxycodone “therapy selection” is determined by a score that goes from 0-12: if a patient receives a score of 0-6=“Poor Responder”; and if a patient receives a score of 7-12=“Good Responder.”

The score is determined by summing the following genetic information shown below in Table 9:

TABLE 9
Oxycodone Genetic Information

| | RS | ANC | ANC | HET | HET | NONA | NONA |
|---|---|---|---|---|---|---|---|
| Gene | Number | Def. | Value | Def. | Value | Def | Value |
| ABCB1 | rs1045642 | CC | 0 | CT | 0 | TT | 2 |
| ABCB1 | rs2032582 | GG | 0 | GA | 0 | AA | 0 |
| ABCB1 | rs2032582 | TA | 1 | GT | 2 | TT | 2 |
| OPRM1 | rs1799971 | AA | 2 | AG | 0 | GG | 0 |
| HT2RA | rs7997012 | GG | 2 | GA | 0 | AA | 2 |
| COMT | rs4680 | GG | 2 | GA | 2 | AA | 2 |
| GABRG2 | rs211014 | CC | 2 | GA | 0 | AA | 2 |

In addition, select CYP haplotype polymorphisms from CYP3A4, CYP3A5 and CYP2D6 are identified as associated with oxycodone risk. The haplotypes from CYP3A4 and CYP2D6 are listed in Table 3 above. In addition, Table 10 below lists the haplotype and scoring associated with CYP3A5 and Table 11 below shows the combination scoring considering CYP3A5 and CYP3A4 together for oxycodone grading purposes. This profile for oxycodone includes an analysis of the enzymes CYP3A4, CYP3A5 and CYP2D6, in which the presence of genetic coding variants indicates a risk factor for oxycodone associated side effects due to a reduction in the enzymes' rate of metabolism. The risk profile combines the evaluation of relevant signalling cascades and metabolizing pathways to provide information regarding oxycodone-induced risk factors for clinical use and management. Physicians may use this test to determine the likelihood of a patient experiencing an oxycodone-related adverse event and/or to assist with prescribing oxycodone at therapeutic doses.

TABLE 10
CYP3A5 Haplotype Polymorphisms

| CYP3A5 | TAG(S) | rs776746 | rs41303343 | rs10264272 | rs55817950 | rs28383479 |
|---|---|---|---|---|---|---|
| *1A | | T | del | C | G | C |
| *1B | | T | del | C | G | C |
| *1C | | T | del | C | G | C |
| *1D | | T | del | C | G | C |
| *1E | | T | del | C | G | C |
| *2 | rs28365083 | T | del | C | G | C |
| *3A | rs776746 | C | del | C | G | C |
| *3B | rs776746 | C | del | C | G | C |
| *3C | rs776746 | C | del | C | G | C |
| *3D | rs776746 | C | del | C | G | C |
| *3E | rs776746 | C | del | C | G | C |
| *3F | rs776746 | C | del | C | G | C |
| *3G | rs776746 | C | del | C | G | C |
| *3H | rs776746 | C | del | C | G | C |
| *3I | rs776746 | C | del | C | G | C |
| *3J | rs776746 | C | del | C | G | C |
| *3K | rs776746 | C | del | C | G | C |
| *3L | rs776746 | C | del | C | G | C |
| *4 | rs56411402 | T | del | C | G | C |
| *5 | | T | del | C | G | C |
| *6 | rs10264272 | T | del | T | G | C |
| *7 | rs41303343 | T | A | C | G | C |
| *8 | rs55817950 | T | del | C | A | C |
| *9 | rs28383479 | T/C | del | C | G | T |

CYP3A5: Star Allele Actvity Scoring

| INCREASED | DECREASED = 0.5 | NORMAL = 1 | NONE = 0 |
|---|---|---|---|
| none | *8 | *1A | *7 |
| | *9 | *1 | *3 |
| | | *2 | *6 |
| | | *4 | |
| | | *5 | |

CYP3A5: Grading based on Activity Score

| | |
|---|---|
| D | 0 = null/null |
| D | 0.5 = null/reduced function |
| C | 1 = reduced/reduced OR normal/null |
| B | 1.5 = reduced/normal |
| B | 2 = normal/normal |

TABLE 11
Combining CYP3A4 and CYP3A5 Haplotype
Polymorphisms for Grading

| CYP3A4/5 | | | | | |
|---|---|---|---|---|---|
| combined | | | | | |
| grade | 3A4 A | 3A4 B | 3A4 C | 3A4 D | 3A4 NA |
| 3A5 B | A | A | B | C | NA |
| 3A5 C | A | B | B | D | NA |
| 3A5 D | A | B | C | D | NA |

Oxycodone dosing recommendation comes from Drug Metabolism (DME) “grades” that are determined using CYP450 SNPs grading algorithms. Oxycodone is metabolized by both CYP3A4/3A5 and CYP2D6 and the dosing recommendations are determined as shown in the Table 10 below.

TABLE 10
Oxycodone Dosing Recommendations
CYP3A4/CYP3A5 Combination**

| | | A | B | C | D |
|---|---|---|---|---|---|
| CYP2D6 | A | This patient may be at risk | This patient is at risk | This patient is at risk | This patient is at risk |
| | | of experiencing an adverse | of experiencing an | of experiencing an | of experiencing an |
| | | drug event due to increased | adverse drug event | adverse drug event | adverse drug event |
| | | metabolism of oxycodone to | with Oxycodone due | with Oxycodone due | with Oxycodone due |
| | | oxymorphone or may have | to increased | to increased | to increased |
| | | insufficient pain relief due to | metabolism to | metabolism to | metabolism to |
| | | increased clearance. | oxymorphone. | oxymorphone. | oxymorphone. |
| | | Consider a lower starting | Consider a lower | Consider a lower | Consider a lower |
| | | dose and monitor closely for | starting dose or | starting dose or select | starting dose or select |
| | | side effects. If pain relief is | select alternative | alternative | alternative |
| | | insufficient and patient is | medication not | medication not | medication not |
| | | tolerating the medication | metabolized by | metabolized by | metabolized by |
| | | well, consider higher | CYP2D6. | CYP2D6. | CYP2D6. |
| | | maintenance dose. | | | |
| | B | This patient may experience | This patient is | This patient is at risk | This patient is at risk |
| | | insufficient pain relief with | predicted to be a | of experiencing an | of experiencing an |
| | | average doses of Oxycodone | normal metabolizer | adverse drug event | adverse drug event |
| | | due to increased clearance. | of Oxycodone. | with Oxycodone due | with Oxycodone due |
| | | Monitor the patient's | Prescribe at | to decreased | to decreased |
| | | analgesic response and | standard label | clearance. Consider a | clearance. Consider a |
| | | consider prescribing an | recommendations. | lower starting dose | lower starting dose |
| | | increased dose if necessary. | | and increase with | and increase with |
| | | | | caution. | caution. |
| | C | This patient may experience | This patient may | This patient is at risk | This patient is at risk |
| | | insufficient pain relief with | experience | of experiencing an | of experiencing an |
| | | average doses of Oxycodone | insufficient pain | adverse drug event | adverse drug event |
| | | due to increased clearance | relief with average | with Oxycodone due | with Oxycodone due |
| | | and decreased metabolism | doses of Oxycodone | to decreased | to decreased |
| | | to oxymorphone. Monitor | due to decreased | clearance or may | clearance or may |
| | | the patient's analgesic | metabolism to | have insufficient pain | have insufficient pain |
| | | response and consider | oxymorphone. | relief due to | relief due to |
| | | prescribing an increased | Monitor the | decreased | decreased |
| | | dose if necessary or select | patient's analgesic | metabolism to | metabolism to |
| | | alternative medication. | response and | oxymorphone. | oxymorphone. |
| | | | consider prescribing | Consider a lower | Consider a lower |
| | | | an increased dose if | starting dose and | starting dose and |
| | | | necessary or select | increase with caution | increase with caution |
| | | | alternative | or select alternative | or select alternative |
| | | | medication. | medication. | medication. |
| | D | This patient may experience | This patient may | This patient is at risk | This patient is at risk |
| | | insufficient pain relief with | experience | of experiencing an | of experiencing an |
| | | average doses of Oxycodone | insufficient pain | adverse drug event | adverse drug event |
| | | due to increased clearance | relief with average | with Oxycodone due | with Oxycodone due |
| | | and decreased metabolism | doses of Oxycodone | to decreased | to decreased |
| | | to oxymorphone. Monitor | due to decreased | clearance or may | clearance or may |
| | | the patient's analgesic | metabolism to | have insufficient pain | have insufficient pain |
| | | response and consider | oxymorphone. | relief due to | relief due to |
| | | prescribing an increased | Monitor the | decreased | decreased |
| | | dose if necessary or select | patient's analgesic | metabolism to | metabolism to |
| | | alternative medication. | response and | oxymorphone. | oxymorphone. |
| | | | consider prescribing | Consider a lower | Consider a lower |
| | | | an increased dose if | starting dose and | starting dose and |
| | | | necessary or select | increase with caution | increase with caution |
| | | | alternative | or select alternative | or select alternative |
| | | | medication. | medication. | medication. |
| | C/D | This patient may experience | This patient may | This patient is at risk | This patient is at risk |
| | | insufficient pain relief with | experience | of experiencing an | of experiencing an |
| | | average doses of Oxycodone | insufficient pain | adverse drug event | adverse drug event |
| | | due to increased clearance | relief with average | with Oxycodone due | with Oxycodone due |
| | | and decreased metabolism | doses of Oxycodone | to decreased | to decreased |
| | | to oxymorphone. Monitor | due to decreased | clearance or may | clearance or may |
| | | the patient's analgesic | metabolism to | have insufficient pain | have insufficient pain |
| | | response and consider | oxymorphone. | relief due to | relief due to |
| | | prescribing an increased | Monitor the | decreased | decreased |
| | | dose if necessary or select | patient's analgesic | metabolism to | metabolism to |
| | | alternative medication. | response and | oxymorphone. | oxymorphone. |
| | | | consider prescribing | Consider a lower | Consider a lower |
| | | | an increased dose if | starting dose and | starting dose and |
| | | | necessary or select | increase with caution | increase with caution |
| | | | alternative | or select alternative | or select alternative |
| | | | medication. | medication. | medication. |
| | B/C/D | This patient is a possible | This patient is a | This patient is at risk | This patient is at risk |
| | | decreased metabolizer of | possible decreased | of experiencing an | of experiencing an |
| | | Oxycodone into | metabolizer of | adverse drug event | adverse drug event |
| | | Oxymorphone. There is a | Oxycodone into | with Oxycodone due | with Oxycodone due |
| | | chance that this patient may | Oxymorphone. | to decreased | to decreased |
| | | experience insufficient pain | There is a chance | clearance or may | clearance or may |
| | | relief. Initiate recommended | that this patient may | possibly have | possibly have |
| | | starting dose and monitor | experience | insufficient pain relief | insufficient pain relief |
| | | closely. If pain relief is | insufficient pain | due to decreased | due to decreased |
| | | insufficient, consider | relief. Initiate | metabolism to | metabolism to |
| | | prescribing an increased | recommended | oxymorphone. | oxymorphone. |
| | | dose if necessary, or select | starting dose and | Consider a lower | Consider a lower |
| | | alternative medication. | monitor closely. If | starting dose and | starting dose and |
| | | | pain relief is | increase with caution | increase with caution |
| | | | insufficient, consider | or select alternative | or select alternative |
| | | | prescribing an | medication. | medication. |
| | | | increased dose if | | |
| | | | necessary. | | |

As shown above, for HTR2A (rs7997012): G/G-A/A is more associated with good response, and G/A is more associated with poor response to oxycodone; COMT (rs4680): A/A-G/A is more associated with good response, and G/G is more associated with poor response; and GABA (rs211014): C/C-A/A is more associated with good response, and C/A is more associated with poor response.

Tramadol

The SNP diploid polymorphisms identified as having a predisposition to response to the opioid tramadol are listed below. In particular, Table 11 identifies the SNP diploid polymorphs associated with tramadol response.

TABLE 11*
Identification of SNP Diploid Polymorphisms-Tramadol

| | | SNP | | | |
|---|---|---|---|---|---|
| No. | rs# | Diploid ID** | Zygosity | DNA Context Sequence for Active SNP(s)*** | SEQ ID |
| 1 | rs1799971 | OPRM1-ANC | homozygous | See above | SEQ ID No: 1 |
| 2 | rs1799971 | OPRM1-HET | heterozygous | See above | SEQ ID No: 2 |
| 3 | rs1799971 | OPRM1-NONA | homozygous | See above | SEQ ID No: 3 |
| 4 | rs140701 | SLC6A4-ANC | homozygous | CACATAAGGTCTTGTGATGAGAATT[G]TAACTGTTGTTGT | SEQ ID No: 28 |
| | | | | GGCTGAGTTTTC | |
| 5 | rs140701 | SLC6A4-HETA4 | heterozygous | CACATAAGGTCTTGTGATGAGAATT[A/G]TAACTGTTGTT | SEQ ID No: 29 |
| | | | | GTGGCTGAGTTTTC | |
| 6 | rs140701 | SLC6A4-NONA | homozygous | CACATAAGGTCTTGTGATGAGAATT[A]TAACTGTTGTTGT | SEQ ID No: 30 |
| | | | | GGCTGAGTTTTC | |
| 7 | rs622342 | SLC22A1_OCT | homozygous | See above | SEQ ID No: 19 |
| | | A > C-ANC | | | |
| 8 | rs622342 | SLC22A1_OCT | heterozygous | See above | SEQ ID No: 20 |
| | | A > C-HET | | | |
| 9 | rs622342 | SLC22A1_OCT | homozygous | See above | SEQ ID No: 21 |
| | | A > C-NONA | | | |
| 10 | rs4680 | COMT-ANC | homozygous | See above | SEQ ID No: 7 |
| 11 | rs4680 | COMT-HET | heterozygous | See above | SEQ ID No: 8 |
| 12 | rs4680 | COMT-NONA | homozygous | See above | SEQ ID No: 9 |

*Unless otherwise indicated, the context sequences are in FASTA format, as presented by NCBI within the rs cluster report
identified by “rs#” in the NCBI SNP reference database accessible at http://www.ncbi.nlm.nih.gov/snp.
**The naming conventions for the SNP Diploid Polymorphisms indicate the diploid is either -ANC (homozygous for the
ancestral SNP), -HET (heterozygous as including one ancestral and one non-ancestral SNP in the diploid), or -NONA
(homozygous for the non-ancestral SNP).
***Brackets (i.e. “[. . .]”) appear within each context sequence to indicate the location (i.e., the “polymorphism marker”
or “marker”) of the polymorphic region in the context sequence.

Studies have been conducted and it has been determined that SNP diploid polymorphisms identified in Table 11 are predictive of a differential predisposition to tramadol response associated with a patient having one or more of SNP diploid polymorphisms. Select SNP diploid polymorphisms in Table 11 are associated with a patient having an elevated tramadol response (i.e., predisposed to having a higher tramadol response).

Tramadol therapy selection is determined by a score that goes from 0-18: if a patient receives a score of 0-4=“Poor Responder”; and if a patient receives a score of 5-8=“Good Responder.” The score is determined by summing the following genetic information shown below in Table 12:

TABLE 12
Tramadol Genetic Information

| | RS | ANC | ANC | HET | HET | NONA | NONA |
|---|---|---|---|---|---|---|---|
| Gene | Number | Def. | Value | Def. | Value | Def | Value |
| OPRM1 | rs1799971 | AA | 2 | AG | 0 | GG | 0 |
| SLC6A4 | rs140701 | GG | 2 | GA | 2 | AA | 0 |
| SLC22A1_OCT | rs622342 | AA | 0 | AC | 2 | CC | 2 |
| A > C | | | | | | | |
| COMT | rs4680 | GG | 0 | GA | 0 | AA | 2 |

In addition, select CYP haplotype polymorphisms from CYP3A4 and CYP2D6 are identified as associated with tramadol risk. The haplotypes from CYP3A4 and CYP2D6 are listed in Table 3 above. This profile for tramadol includes an analysis of the enzymes CYP3A4 and CYP2D6, in which the presence of genetic coding variants indicates a risk factor for tramadol associated side effects due to a reduction in the enzymes' rate of metabolism. The risk profile combines the evaluation of relevant signalling cascades and metabolizing pathways to provide information regarding tramadol-induced risk factors for clinical use and management. Physicians may use this test to determine the likelihood of a patient experiencing an tramadol-related adverse event and/or to assist with prescribing oxycodone at therapeutic doses.

Tramadol Dosing Recommendation comes from Drug Metabolism (DME) “grades” that are determined using CYP450 SNP grading algorithms and an analysis of the rs622342 (SLC22A1) polymorphism. Tramadol is metabolized by both CYP3A4 and CYP2D6 and is a substrate of the SLC22A1 transporter. The dosing recommendations are determined as shown in the Table 13 below.

TABLE 13
Tramadol Dosing Recommendations
CYP3A4

| | | A | B | C | D |
|---|---|---|---|---|---|
| CYP2D6 | A | This patient is at | This patient is at | This patient is | This patient is |
| | | risk of | risk of | at risk of | at risk of |
| | | experiencing an | experiencing an | experiencing an | experiencing an |
| | | adverse drug | adverse drug | adverse drug | adverse drug |
| | | event (ADE) | event (ADE) | event (ADE) | event (ADE) |
| | | with tramadol. | with tramadol. | with tramadol. | with tramadol. |
| | | According to | According to | According to | According to |
| | | published | published | published | published |
| | | guidelines, | guidelines, | guidelines, | guidelines, |
| | | consider a 30% | consider a 30% | consider a 30% | consider a 30% |
| | | decreased dose | decreased dose | decreased dose | decreased dose |
| | | and be alert for | and be alert for | and be alert for | and be alert for |
| | | ADEs, or use an | ADEs, or use an | ADEs, or use an | ADEs, or use an |
| | | alternative to | alternative to | alternative to | alternative to |
| | | tramdol that is | tramdol that is | tramdol that is | tramdol that is |
| | | not oxycodone | not oxycodone | not oxycodone | not oxycodone |
| | | or codeine, | or codeine, | or codeine, | or codeine. |
| | B and SLC2A1 | This patient is | This patient is | This patient is | This patient |
| | OCT1 = A/A | predicted to be a | predicted to be a | predicted to be | may be at risk |
| | | normal | normal | a normal | of experiencing |
| | | metabolizer of | metabolizer of | metabolizer of | an adverse drug |
| | | tramadol. | tramadol. | tramadol. | event with |
| | | Prescribe at | Prescribe at | Prescribe at | tramadol. |
| | | standard label | standard label | standard label | Consider |
| | | recommendations | recommendations | recommendations. | lowering the |
| | | | | | dose of the |
| | | | | | medication. |
| | B and | This patient is | This patient may | This patient | This patient |
| | SLC2A1_OCT1 = | predicted to be a | be at risk of | may be at risk | may be at risk |
| | A/C or C/C | normal | experiencing an | of experiencing | of experiencing |
| | | metabolizer of | adverse drug | an adverse drug | an adverse drug |
| | | tramadol. | event with | event with | event with |
| | | Prescribe at | tramadol. | tramadol. | tramadol. |
| | | standard label | Consider | Consider | Consider |
| | | recommendations | lowering the | lowering the | lowering the |
| | | | dose of the | dose of the | dose of the |
| | | | medication. | medication. | medication. |
| | C | This patient may | This patient may | This patient | This patient |
| | | experience | experience | may experience | may experience |
| | | insufficient pain | insufficient pain | insufficient pain | insufficient pain |
| | | relief with | relief with | relief with | relief with |
| | | average doses of | average doses of | average doses | average doses |
| | | tramadol | tramadol. | of tramadol. | of tramadol |
| | | Monitor the | Monitor the | Monitor the | and/or is at |
| | | patient's | patient's | patient's | increased risk |
| | | analgesic | analgesic | analgesic | for experiencing |
| | | response and | response and | response and | an adverse drug |
| | | consider | consider | consider | event. Consider |
| | | prescribing an | prescribing an | prescribing an | choosing an |
| | | increased dose if | increased dose if | increased dose | alternative |
| | | necessary. | necessary. | if necessary | medication. |
| | D | This patient may | This patient may | This patient | This patient |
| | | experience | experience | may experience | may experience |
| | | insufficient pain | insufficient pain | insufficient pain | insufficient pain |
| | | relief with | relief with | relief with | relief with |
| | | average doses of | average doses of | average doses | average doses |
| | | tramadol | tramadol | of tramadol | of tramadol |
| | | Monitor the | Monitor the | and/or is at | and/or is at |
| | | patient's | patient's | increased risk | increased risk |
| | | analgesic | analgesic | for experiencing | for experiencing |
| | | response and | response and | an adverse drug | an adverse drug |
| | | consider | consider | event. Consider | event. Consider |
| | | prescribing an | prescribing an | choosing an | choosing an |
| | | increased dose if | increased dose if | alternative | alternative |
| | | necessary. | necessary. | medication. | medication. |
| | B/C/D | This patient is a | This patient is a | This patient is a | This patient |
| | | possible | possible | possible | may experience |
| | | decreased | decreased | decreased | insufficient pain |
| | | metabolizer of | metabolizer of | metabolizer of | relief with |
| | | tramadol into its | tramadol into its | tramadol into its | average doses |
| | | metabolite. | metabolite. | metabolite. | of tramadol |
| | | There is a | There is a chance | There is a | and/or is at |
| | | chance that this | that this patient | chance that this | increased risk |
| | | patient may | may experience | patient may | for experiencing |
| | | experience | insufficient pain | experience | an adverse drug |
| | | insufficient pain | relief. Monitor | insufficient pain | event. Consider |
| | | relief. Monitor | the patient's | relief. Monitor | choosing an |
| | | the patient's | analgesic | the patient's | alternative |
| | | analgesic | response and | analgesic | medication. |
| | | response and | increase dose if | response and | |
| | | increase dose if | necessary. | increase dose if | |
| | | necessary. | | necessary. | |
| | C/D | This patient may | This patient may | This patient | This patient |
| | | experience | experience | may experience | may experience |
| | | insufficient pain | insufficient pain | insufficient pain | insufficient pain |
| | | relief with | relief with | relief with | relief with |
| | | average doses of | average doses of | average doses | average doses |
| | | tramadol | tramadol | of tramadol | of tramadol |
| | | Monitor the | Monitor the | and/or is at | and/or is at |
| | | patient's | patient's | increased risk | increased risk |
| | | analgesic | analgesic | of an adverse | of an adverse |
| | | response and | response and | drug event, | drug event. |
| | | consider | consider | Consider | Consider |
| | | prescribing an | prescribing an | choosing an | choosing an |
| | | increased dose or | increased dose or | alternative | alternative |
| | | choosing an | choosing an | medication. | medication. |
| | | alternative | alternative | | |
| | | medication if | medication if | | |
| | | necessary. | necessary. | | |

As shown above, for COMT (rs4680): GG/GA is more associated with poor response, and A/A is more associated with good response.

Tramadol is a synthetic opioid, centrally-acting analgesic for the treatment of moderate to severe pain and is mainly metabolized by cytochrome P450 CYP2D6. Tramadol is generally considered to be devoid of any serious adverse effects of traditional opioid receptor agonists, such as respiratory depression and drug dependence. CYP2D6 is a saturable enzyme responsible for metabolism of approximately 25% of known pharmaceuticals and plays a significant role in tramadol analgesic efficacy. In studies, polymorphisms in OCT1 were found to influence the pharmacokinetics of O-desmethyltramadol, presumably by affecting its uptake into liver cells, and thus may modulate the efficacy of tramadol treatment. The metabolic ratio of ODT/TRA was found to be used for estimation of the time of drug intake, but only when the CYP2D6 genotype is known and taken into consideration. In patients with excessive morphinomimetic effects, levels of tramadol could be measured, ideally combined with CYP2D6 genotyping, to identify individuals at risk of tramadol-related cardiotoxicity. Tramadol treatment can be optimized in at-risk individuals, consequently improving patient outcome and safety. CYP2D6 drug-drug interactions appear to change effectiveness of commonly prescribed drugs, such as Tramadol. Drug-drug interaction should be considered prior to prescribing CYP2D6 drugs. The response profile predicts a patient's genetic response to tramadol medications, and will advise the prescribing physician any potential adverse drug events, and can assist the physician with properly prescribing tramadol at optimal doses for each patient's individual needs.

Opioid response assessment relys on non-invasive measures of biological pathways. The use of pharmacogenetic testing provides a quick and easy evaluation of opioid response associated with opioid use, in addition to providing an avenue for identification of new measures that may lead to increased accuracy in patient risk stratification. With a simple buccal swab, the risk test investigates potential gene-drug interactions analyzing enzyme targets of opioids. Any human sample providing isolatable genomic DNA is acceptable for this test; examples are: buccal swabs, blood, urine, or tissue samples. Using this approach, guidance for the rational use of opioid therapy and clinical protocals can be achieved. For example, by identifying patients more likely to be good vs. poor responders; and providing alternative measures to control pain in patients with a poor likelihood of response. Alternative pain control measures to be considered based on the results of this test may lead to better patient outcomes, decreased use of suboptimal medications, and shorter duration of therapy and lower costs. Additionally, a characterization of a patient's metabolic profile for opioid response would add crucial information to a patient's clinical care as well.

Detection of point mutations or other types of the allelic variants disclosed herein, can be accomplished several ways known in the art, such as by molecular cloning of the specified allele and subsequent sequencing of that allele using techniques known in the art. Alternatively, the gene sequences can be amplified directly from a genomic DNA preparation from the DNA sample using PCR, and the sequence composition is determined from the amplified product. As described more fully below, numerous methods are available for analyzing a subject's DNA for mutations at a given genetic locus such as the gene of interest.

One such detection method is allele specific hybridization using probes overlapping the polymorphic region and having, for example, about 5, or alternatively 10, or alternatively 20, or alternatively 25, or alternatively 30 nucleotides around the polymorphic region. In another embodiment, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described, e.g., in Cronin et al. (1996) Human Mutation 7:244.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell. Probes 6:1).

If the polymorphic region is located in the coding region of the gene of interest, yet other methods than those described above can be used for determining the identity of the allelic variant according to methods known in the art.

The genotype information obtained from analyzing a sample of a patient's genetic material may be utilized, according to the principles of the invention, to predict whether a patient has a level of risk associated with poor opioid response. The risk may be associated with a side effect the patient may be susceptible to developing, an efficacy of the drug to the patient specifically or some combination thereof. The genotype information of the patient may be combined with demographic information about the patient as described above.

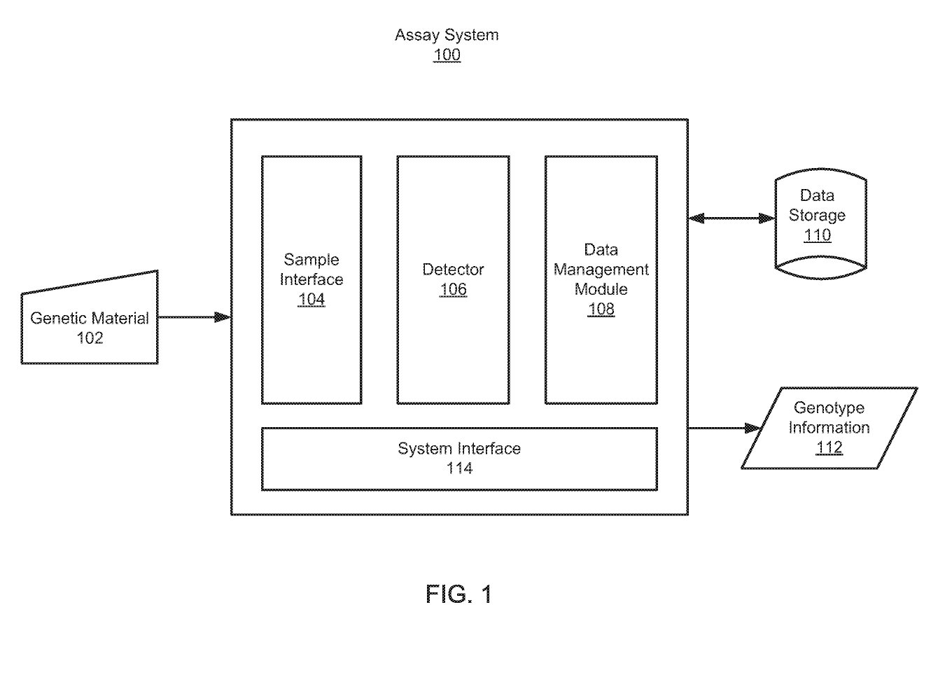

Referring to FIG. 1, depicted is an assay system 100. An assay system, such as assay system 100, may access or receive a genetic material, such as genetic material 102. The sample of genetic material 102 can be obtained from a patient by any suitable manner. The sample may be isolated from a source of a patient's DNA, such as saliva, buccal cells, hair roots, blood, cord blood, amniotic fluid, interstitial fluid, peritoneal fluid, chorionic villus, semen, or other suitable cell or tissue sample. Methods for isolating genomic DNA from various sources are well-known in the art. Also contemplated are non-invasive methods for obtaining and analyzing a sample of genetic material while still in situ within the patient's body.

The genetic material 102 may be received through a sample interface, such as sample interface 104 and detected using a detector, such as detector 106. A polymorphism may be detected in the sample by any suitable manner known in the art. For example, the polymorphism can be detected by techniques, such as allele specific hybridization, allele specific oligonucleotide ligation, primer extension, minisequencing, mass spectroscopy, heteroduplex analysis, single strand conformational polymorphism (SSCP), denaturing gradient gel electrophoresis (DGGE), oligonucleotide microarray analysis, temperature gradient gel electrophoresis (TGGE), and combinations thereof to produce an assay result. The assay result may be processed through a data management module, such as data management module 108, to produce genotype information 112. The genotype information 112 may include an assay result on whether the patients has a genotype including one or more of the allelic variants listed in Tables I and 3 above. The genotype information 112 may be stored in data storage 110 or transmitted to another system or entity via a system interface 114.

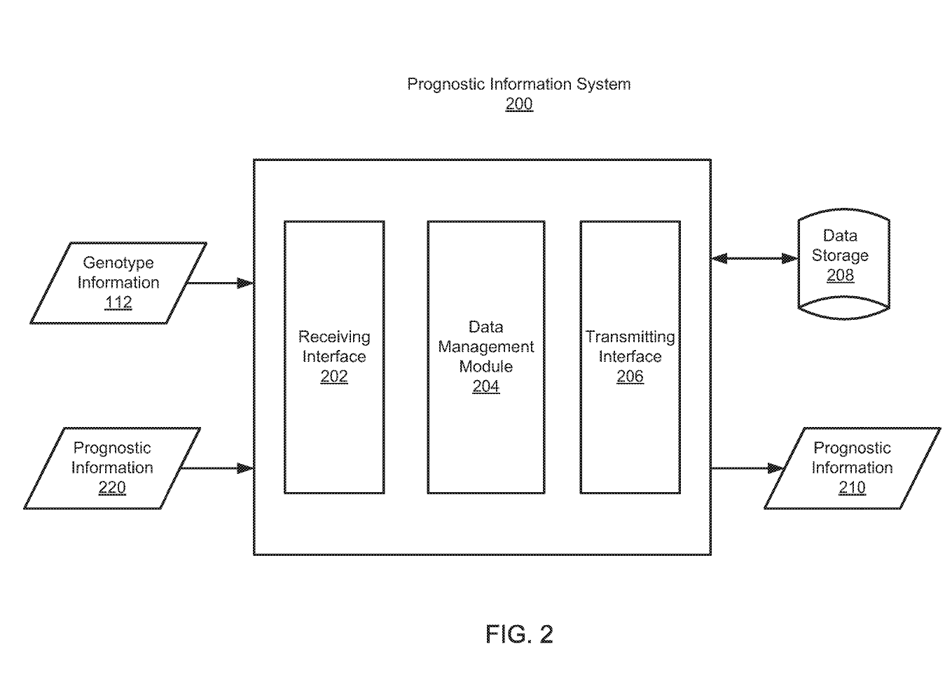

Referring to FIG. 2, depicted is a prognostic information system 200. The prognostic information system 200 may be remotely located away from the assay system 100 or operatively connected with it in an integrated system. The prognostic information system 200 receives the genotype information 112 through a receiving interface 202 for processing at a data management module 204 to generate prognostic information 210. The data management module 204 may utilize one or more algorithms described in greater detail below to generate prognostic information 210. The prognostic information 210 may be stored in data storage 208 or transmitted via a transmitting interface 206 to another system or entity. The transmitting interface 206 may be the same or different as the receiving interface 202. Furthermore, the system 200 may receive prognostic information 220 prepared by another system or entity. Prognostic information may be utilized, in addition to or in the alternative, to genotype information 112 in generating prognostic information 210.

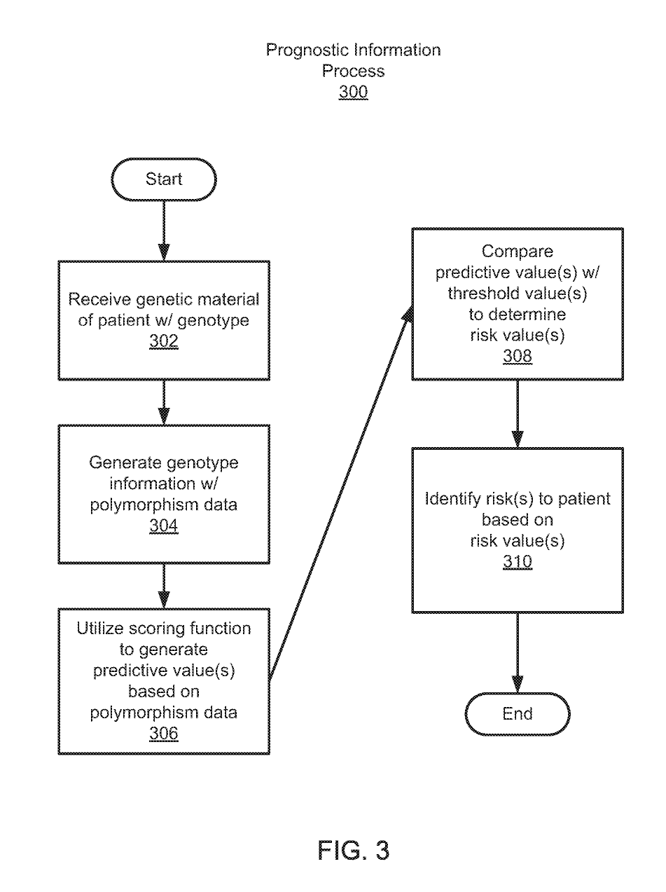

Referring to FIG. 3, depicted is a prognostic information process 300 which may be utilized for preparing information, such as genotype information 112 and prognostic information 210, utilizing an assay system, such as assay system 100 and/or a prognostic information system, such as prognostic information system 200, according to an embodiment. The steps of process 300, and other methods described herein, are described by way of example with the assay system 100 and the prognostic information system 200. The process 300 may be performed with other systems as well.

After process start, at step 302, a sample of genetic material of a patient is obtained as it is received at the sample interface 106. The sample interface can be any type of receptacle for holding or isolating the genetic material 102 for assay testing.

At step 304, the genetic material 102 is tested utilizing the detector 106 in assay system 100 to generate genotype information 112. The detector 106 may employ any of the assay methodologies described above to identify allelic variants in the genetic material 102 and generate the genotype information 112 including polymorphism data associated with one or more of the DNA polymorphisms described above in Tables 1 and 3. The data management module 108, utilizing a processor in an associated platform such as described below, may store the genotype information 112 on the data storage 110 and/or transmit the genotype information 112 to another entity or system, such as prognostic information system 200 where it is received at receiving interface 202 for analysis.

At step 306, the genotype information 112 can be analyzed utilizing a processor in an associated platform, such as described below, by using an algorithm which may be programmed for processing through data management module 204. The algorithm may utilize a scoring function to generate predictive values based on the polymorphism data in the genotype information 112. Different algorithms may be utilized to assign predictive values and aggregate values.

For example, an additive effect algorithm may be utilized to generate an analysis of a patient's genetic predisposition and their demographic phenotype predisposition to opioid response. In the additive effect algorithm, polymorphism data of the genotype information obtained from analyzing a patient's genetic material is utilized to indicate the active polymorphisms identified from a patient's genotype information. A tested polymorphism may be determined to be (1) absent or present in either (2) a heterozygous or (3) a homozygous variant in the patient's genotype. According to the additive effect algorithm, the polymorphisms identified from a patient's genotype information and demographic phenotype are each assigned a real value, such as an Odds Ratio (OR) or a parameter score, depending on which polymorphisms appears in the patient's genotype and demographic information.

To gather data for the algorithm, one or more of the SNP Diploid Polymorphisms, such as those listed in Tables 1 and 3, may be tested and/or analyzed to produce one or more values associated with the presence or absence of the SNP Diploid Polymorphisms. Other factors, such as other SNP Diploid Polymorphisms, other demographic phenotypes may also be tested and/or analyzed to produce one or more values associated with the presence or absence of the other SNP Diploid Polymorphisms and other demographic phenotypes.

The values gathered are based on results of the various tests and data gathered and/or determined. The values may be factored into an algorithm to score a subject's opioid response based on the subject's genetic information and/or non-genetic characteristics or phenotypes. The algorithm may compute a composite score based on the results of individual tests. The composite score may be calculated based on an additive analysis of the individual scores which may be compared with a threshold value for determining opioid response based on the additive score. In addition or in the alternative, more complex functions may be utilized to process the values developed from the testing results, such as utilizing one or more weighting factor(s) applied to one or more of the individual values based on various circumstances, such as if a subject was tested using specific equipment, a temporal condition, etc.

In all of the preceding examples, the predictive values and aggregate values generated are forms of prognostic information 210.

At step 310, the result of the comparison obtained in step 308 generates a second form of prognostic information 220. For example, (a) if the determined sum is higher than the threshold value, it can be predicted that the patient is at an elevated risk for poor opioid response associated with prescribing the patient an opioid medication; (b) if the determined sum is at or near the threshold value, it can be predicted that the patient is at a moderate risk for poor opioid response; and (c) if the determined sum is below the threshold value, it can be predicted that the patient is at a low risk for poor opioid response.

Also at step 310, the data management module 205 in the prognostic information system 200 identifies a risk to a patient by executing an algorithm, such as the additive effect algorithm described above, and communicating the generated prognostic information 210. The data management module 204, utilizing a processor in an associated platform such as described below, may store the prognostic information 210 on the data storage 208 and/or transmit the prognostic information 210 to another entity or system prior to end of the prognostic information process 300. Other algorithms may also be used in a similar manner to generate useful forms of prognostic information for determining treatment options for a patient.

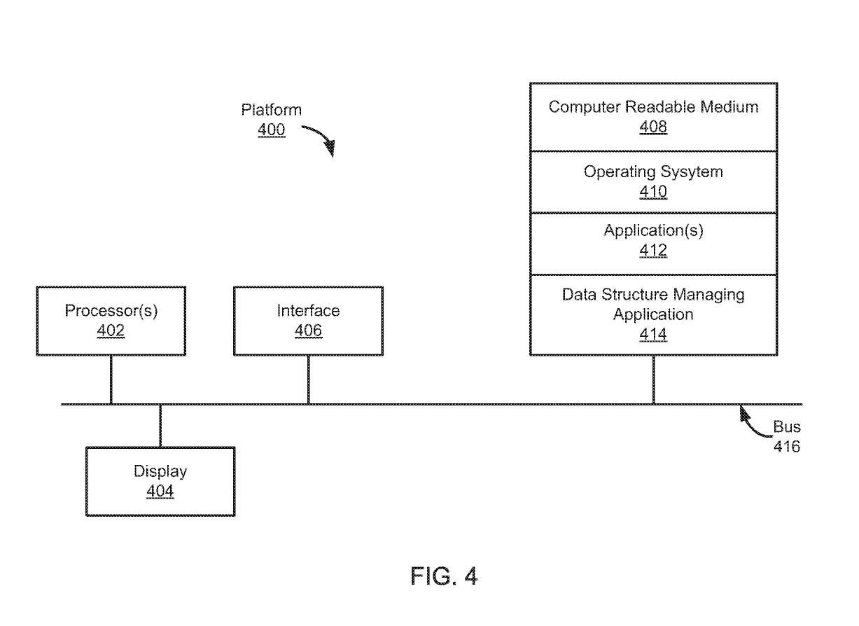

Referring to FIG. 4, there is shown a platform 400, which may be utilized as a computing device in a prognostic information system, such as prognostic information system 200, or an assay system, such as assay system 100. It is understood that the depiction of the platform 400 is a generalized illustration and that the platform 400 may include additional components and that some of the components described may be removed and/or modified without departing from a scope of the platform 400.

The platform 400 includes processor(s) 402, such as a central processing unit; a display 404, such as a monitor; an interface 406, such as a simple input interface and/or a network interface to a Local Area Network (LAN), a wireless 802.11x LAN, a 3G or 4G mobile WAN or a WiMax WAN; and a computer-readable medium (CRM) 408. Each of these components may be operatively coupled to a bus 416. For example, the bus 416 may be an EISA, a PCI, a USB, a FireWire, a NuBus, or a PDS.

A CRM, such as CRM 408 may be any suitable medium which participates in providing instructions to the processor(s) 402 for execution. For example, the CRM 408 may be non-volatile media, such as an optical or a magnetic disk; volatile media, such as memory; and transmission media, such as coaxial cables, copper wire, and fiber optics. Transmission media can also take the form of acoustic, light, or radio frequency waves. The CRM 408 may also store other instructions or instruction sets, including word processors, browsers, email, instant messaging, media players, and telephony code.

The CRM 408 may also store an operating system 410, such as MAC OS, MS WINDOWS, UNIX, or LINUX; application(s) 412, such as network applications, word processors, spreadsheet applications, browsers, email, instant messaging, media players such as games or mobile applications (e.g., “apps”); and a data structure managing application 414. The operating system 410 may be multi-user, multiprocessing, multitasking, multithreading, real-time and the like. The operating system 410 may also perform basic tasks such as recognizing input from the interface 406, including from input devices, such as a keyboard or a keypad; sending output to the display 404 and keeping track of files and directories on CRM 408; controlling peripheral devices, such as disk drives, printers, image capture devices; and for managing traffic on the bus 416. The applications 412 may include various components for establishing and maintaining network connections, such as code or instructions for implementing communication protocols including those such as TCP/IP, HTTP, Ethernet, USB, and FireWire.

A data structure managing application, such as data structure managing application 414 provides various code components for building/updating a computer-readable system architecture, such as for a non-volatile memory, as described above. In certain examples, some or all of the processes performed by the data structure managing application 412 may be integrated into the operating system 410. In certain examples, the processes may be at least partially implemented in digital electronic circuitry, in computer hardware, firmware, code, instruction sets, or any combination thereof.

Although described specifically throughout the entirety of the disclosure, the representative examples have utility over a wide range of applications, and the above discussion is not intended and should not be construed to be limiting. The terms, descriptions and figures used herein are set forth by way of illustration only and are not meant as limitations. Those skilled in the art recognize that many variations are possible within the spirit and scope of the principles of the invention. While the examples have been described with reference to the figures, those skilled in the art are able to make various modifications to the described examples without departing from the scope of the following claims, and their equivalents.

What is claimed is:

1. A method comprising facilitating a processing of and/or processing (1) data and/or (2) information and/or (3) at least one signal, the (1) data and/or (2) information and/or (3) at least one signal based, at least in part, on the following:
determining patient information, including DNA information, associated with a human subject;
determining from the DNA information whether a subject genotype of the human subject includes one or more SNP diploid polymorphisms by detecting, utilizing a detection technology and the DNA information, a presence or absence of the one or more SNP diploid polymorphisms in the subject genotype,
wherein each SNP diploid polymorphism of the one or more SNP diploid polymorphisms includes a combination of two SNP alleles associated with one SNP location,
wherein the one or more SNP diploid polymorphisms are selected from the SNP diploid group:
OPRM1-ANC, OPRM1-HET, and OPRM1-NONA in the OPRM1 gene,
SLC6A3-ANC, SLC6A3-HET, and SLC6A3-NONA in the SLC6A3 gene,
COMT-ANC, COMT-HET, and COMT-NONA in the COMT gene,
ABCB1(C3435T)-ANC, ABCB1(C3435T)-HET, and ABCB1(C3435T)-NONA in the ABCB1 gene,
ABCB1(C1236T)-ANC, ABCB1(C1236T)-HET, and ABCB1(C1236T)-NONA in the ABCB1 gene,
ABCB1(G2677A/T)-ANC, ABCB1(G2677A/T)-HET, and ABCB1(G2677A/T)-NONA in the ABCB1 gene,
SLC22A1 OCT A>C-ANC, SLC22A 1_OCT A>C-HET, and SLC22A 1_OCT A>C-NONA in the SLC22A 1_OCT A>C-gene,
HTR2A-ANC, HT2RA-HET, and HT2RA-NONA in the HT2RA gene,
GABRG2-ANC, GABRG2-HET, and GABRG2-NONA in the GABRG2 gene, and
SLC6A4-ANC, SLC6A4-HET, and SLC6A4-NONA in the SLC6A4 gene; and
determining an opioid response associated with the human subject based, at least in part, on the presence or absence of the one or more SNP diploid polymorphisms in the subject genotype.

2. A method of claim 1, wherein the (1) data and/or (2) information and/or (3) at least one signal are further based, at least in part, on the following:
determining from the DNA information whether a subject genotype of the human subject includes at least two CYP haplotype polymorphisms by detecting, utilizing a detection technology and the DNA information, a presence or absence of the at least two CYP haplotype polymorphisms in the subject genotype,
wherein at least one or more CYP haplotype polymorphisms are selected from CYP3A4 or CYP3A5 star alleles,
wherein at least one or more CYP haplotype polymorphisms are selected from CYP2D6 star alleles,
wherein the method for determining the opioid response associated with the human subject, is an ex vivo method.

3. A method of claim 1, wherein the (1) data and/or (2) information and/or (3) at least one signal are further based, at least in part, on the following:
determining a comparing of a region, including the one or more SNP diploid polymorphisms, of the subject genotype with a corresponding region of a predetermined reference genotype, wherein characteristics of the corresponding region of the reference genotype are based upon a predetermined population norm;
determining prognostic information associated with the human subject based on the determined opioid response; and
determining a therapy for the human subject based on the determined prognostic information associated with the human subject.

4. A method of claim 1, wherein the one or more SNP diploid polymorphisms include at least three SNP diploid polymorphisms from the SNP diploid group.

5. A method of claim 1, wherein the one or more SNP diploid polymorphisms include at least four SNP diploid polymorphisms from the SNP diploid group.

6. A method of claim 1, wherein the one or more SNP diploid polymorphisms include at least five SNP diploid polymorphisms from the SNP diploid group.

7. A method of claim 1, wherein the one or more SNP diploid polymorphisms include at least eleven SNP diploid polymorphisms from the SNP diploid group.

8. An apparatus comprising:
at least one processor; and
at least one memory including computer program code for one or more programs,
the at least one memory and the computer program code configured to, with the at least one processor, cause the apparatus to perform at least the following,
determine patient information, including DNA information, associated with a human subject;
determine from the DNA information whether a subject genotype of the human subject includes one or more SNP diploid polymorphisms by detecting, utilizing a detection technology and the DNA information, a presence or absence of the one or more SNP diploid polymorphisms in the subject genotype,
wherein each SNP diploid polymorphism of the one or more SNP diploid polymorphisms includes a combination of two SNP alleles associated with one SNP location,
wherein the one or more SNP diploid polymorphisms are selected from the SNP diploid group:
OPRM1-ANC, OPRM1-HET, and OPRM1-NONA in the OPRM1 gene,
SLC6A3-ANC, SLC6A3-HET, and SLC6A3-NONA in the SLC6A3 gene,
COMT-ANC, COMT-HET, and COMT-NONA in the COMT gene,
ABCB1(C3435T)-ANC, ABCB1(C3435T)-HET, and ABCB1(C3435T)-NONA in the ABCB1 gene,
ABCB1 (C1236T)-ANC, ABCB1 (C1236T)-HET, and ABCB1(C1236T)-NONA in the ABCB1 gene,
ABCB1(G2677A/T)-ANC, ABCB1(G2677A/T)-HET, and ABCB1(G2677A/T)-NONA in the ABCB1 gene,
SLC22A1 OCT A>C-ANC, SLC22A 1_OCT A>C-HET, and SLC22A 1_OCT A>C-NONA in the SLC22A 1_OCT A>C-gene,
HTR2A-ANC, HT2RA-HET, and HT2RA-NONA in the HT2RA gene,
GABRG2-ANC, GABRG2-HET, and GABRG2-NONA in the GABRG2 gene, and
SLC6A4-ANC, SLC6A4-HET, and SLC6A4-NONA in the SLC6A4 gene;
determine an opioid response associated with the human subject based, at least in part, on the presence or absence of the one or more SNP diploid polymorphisms in the subject genotype.

9. An apparatus of claim 8, wherein the (1) data and/or (2) information and/or (3) at least one signal are further based, at least in part, on the following:
determining from the DNA information whether a subject genotype of the human subject includes at least two CYP haplotype polymorphisms by detecting, utilizing a detection technology and the DNA information, a presence or absence of the at least two CYP haplotype polymorphisms in the subject genotype,
wherein at least one or more CYP haplotype polymorphisms are selected from CYP3A4 or CYP3A5 star alleles,
wherein at least one or more CYP haplotype polymorphisms are selected from CYP2D6 star alleles,
wherein the method for determining the opioid response associated with the human subject, is an ex vivo method.

10. An apparatus of claim 8, wherein the apparatus is further caused to:
determine a comparing of a region, including the one or more SNP diploid polymorphisms, of the subject genotype with a corresponding region of a predetermined reference genotype,
wherein characteristics of the corresponding region of the reference genotype are based upon a predetermined population norm;
determine prognostic information associated with the human subject based on the determined opioid response; and
determine a therapy for the human subject based on the determined prognostic information associated with the human subject.

11. An apparatus of claim 8, wherein the one or more SNP diploid polymorphisms include at least three SNP diploid polymorphisms from the SNP diploid group.

12. An apparatus of claim 11, wherein the one or more SNP diploid polymorphisms include at least four SNP diploid polymorphisms from the SNP diploid group.

13. An apparatus of claim 8, wherein the one or more SNP diploid polymorphisms include at least five SNP diploid polymorphisms from the SNP diploid group.

14. An apparatus of claim 8, wherein the one or more SNP diploid polymorphisms include at least eleven SNP diploid polymorphisms from the SNP diploid group.

15. A non-transitory computer readable medium storing computer readable instructions that when executed by at least one processor perform a method, the method comprising facilitating a processing of and/or processing (1) data and/or (2) information and/or (3) at least one signal, the (1) data and/or (2) information and/or (3) at least one signal based, at least in part, on the following:
determining patient information, including DNA information, associated with a human subject;
determining from the DNA information whether a subject genotype of the human subject includes one or more SNP diploid polymorphisms by detecting, utilizing a detection technology and the DNA information, a presence or absence of the one or more SNP diploid polymorphisms in the subject genotype,
wherein each SNP diploid polymorphism of the one or more SNP diploid polymorphisms includes a combination of two SNP alleles associated with one SNP location,
wherein the one or more SNP diploid polymorphisms are selected from the SNP diploid group:
OPRM1-ANC, OPRM1-HET, and OPRM1-NONA in the OPRM1 gene,
SLC6A3-ANC, SLC6A3-HET, and SLC6A3-NONA in the SLC6A3 gene,
COMT-ANC, COMT-HET, and COMT-NONA in the COMT gene,
ABCB1(C3435T)-ANC, ABCB1(C3435T)-HET, and ABCB1(C3435T)-NONA in the ABCB1 gene,
ABCB1 (C1236T)-ANC, ABCB1 (C1236T)-HET, and ABCB1(C1236T)-NONA in the ABCB1 gene,
ABCB1(G2677A/T)-ANC, ABCB1(G2677A/T)-HET, and ABCB1(G2677A/T)-NONA in the ABCB1 gene,
SLC22A1 OCT A>C-ANC, SLC22A 1_OCT A>C-HET, and SLC22A 1_OCT A>C-NONA in the SLC22A 1_OCT A>C-gene,
HTR2A-ANC, HT2RA-HET, and HT2RA-NONA in the HT2RA gene,
GABRG2-ANC, GABRG2-HET, and GABRG2-NONA in the GABRG2 gene, and
SLC6A4-ANC, SLC6A4-HET, and SLC6A4-NONA in the SLC6A4 gene; and
determining an opioid response associated with the human subject based, at least in part, on the presence or absence of the one or more SNP diploid polymorphisms in the subject genotype.

16. A computer readable medium of claim 15, wherein the (1) data and/or (2) information and/or (3) at least one signal are further based, at least in part, on the following:
determining from the DNA information whether a subject genotype of the human subject includes at least two CYP haplotype polymorphisms by detecting, utilizing a detection technology and the DNA information, a presence or absence of the at least two CYP haplotype polymorphisms in the subject genotype,
wherein at least one or more CYP haplotype polymorphisms are selected from CYP3A4 or CYP3A5 star alleles,
wherein at least one or more CYP haplotype polymorphisms are selected from CYP2D6 star alleles,
wherein the method for determining the opioid response associated with the human subject, is an ex vivo method.

17. A computer readable medium of claim 15, wherein the (1) data and/or (2) information and/or (3) at least one signal are further based, at least in part, on the following:
determining a comparing of a region, including the one or more SNP diploid polymorphisms, of the subject genotype with a corresponding region of a predetermined reference genotype,
wherein characteristics of the corresponding region of the reference genotype are based upon a predetermined population norm;
determining prognostic information associated with the human subject based on the determined opioid response; and
determining a therapy for the human subject based on the determined prognostic information associated with the human subject.

18. A computer readable medium of claim 15, wherein the one or more SNP diploid polymorphisms include at least three SNP diploid polymorphisms from the SNP diploid group.

19. A computer readable medium of claim 15, wherein the one or more SNP diploid polymorphisms include at least five SNP diploid polymorphisms from the SNP diploid group.

20. A computer readable medium of claim 15, wherein the one or more SNP diploid polymorphisms include at least eleven SNP diploid polymorphisms from the SNP diploid group.


SEQUENCE LISTING (30 sequences)

SEQ ID NO: 1 - DNA, 52 bp, Homo sapiens
ggtcaacttgtcccacttagatggcaacctgtccgacccatgcggtccgaac

SEQ ID NO: 2 - DNA, 52 bp, Homo sapiens
ggtcaacttgtcccacttagatggcracctgtccgacccatgcggtccgaac

SEQ ID NO: 3 - DNA, 52 bp, Homo sapiens
ggtcaacttgtcccacttagatggcgacctgtccgacccatgcggtccgaac

SEQ ID NO: 4 - DNA, 51 bp, Homo sapiens
agtgcccctggggcagcctcagagccgggagcagggagcagggagggaggg

SEQ ID NO: 5 - DNA, 51 bp, Homo sapiens
agtgcccctggggcagcctcagagcygggagcagggagcagggagggaggg

SEQ ID NO: 6 - DNA, 51 bp, Homo sapiens
agtgcccctggggcagcctcagagctgggagcagggagcagggagggaggg

SEQ ID NO: 7 - DNA, 51 bp, Homo sapiens
ccagcggatggtggatttcgctggcgtgaaggacaaggtgtgcatgcctga

SEQ ID NO: 8 - DNA, 51 bp, Homo sapiens
ccagcggatggtggatttcgctggcrtgaaggacaaggtgtgcatgcctga

SEQ ID NO: 9 - DNA, 51 bp, Homo sapiens
ccagcggatggtggatttcgctggcatgaaggacaaggtgtgcatgcctga

SEQ ID NO: 10 - DNA, 51 bp, Homo sapiens
gccgggtggtgtcacaggaagagatcgtgagggcagcaaaggaggccaaca

SEQ ID NO: 11 - DNA, 51 bp, Homo sapiens
gccgggtggtgtcacaggaagagathgtgagggcagcaaaggaggccaaca

SEQ ID NO: 12 - DNA, 51 bp, Homo sapiens
gccgggtggtgtcacaggaagagattgtgagggcagcaaaggaggccaaca

SEQ ID NO: 13 - DNA, 51 bp, Homo sapiens
actcgtcctggtagatcttgaagggcctgaacctgaaggtgcagagtgggc

SEQ ID NO: 14 - DNA, 51 bp, Homo sapiens
actcgtcctggtagatcttgaagggyctgaacctgaaggtgcagagtgggc

SEQ ID NO: 15 - DNA, 51 bp, Homo sapiens
actcgtcctggtagatcttgaagggtctgaacctgaaggtgcagagtgggc

SEQ ID NO: 16 - DNA, 51 bp, Homo sapiens
gaaagataagaaagaactagaaggtgctgggaaggtgagtcaaactaaata

SEQ ID NO: 17 - DNA, 51 bp, Homo sapiens
gaaagataagaaagaactagaaggtdctgggaaggtgagtcaaactaaata

SEQ ID NO: 18 - DNA, 51 bp, Homo sapiens
gaaagataagaaagaactagaaggttctgggaaggtgagtcaaactaaata

SEQ ID NO: 19 - DNA, 49 bp, Homo sapiens
ttcttcaaatttgatgaaaacttcaaatacatagatctaacaatctaat

SEQ ID NO: 20 - DNA, 49 bp, Homo sapiens
ttcttcaaatttgatgaaaacttcmaatacatagatctaacaatctaat

SEQ ID NO: 21 - DNA, 49 bp, Homo sapiens
ttcttcaaatttgatgaaaacttccaatacatagatctaacaatctaat

SEQ ID NO: 22 - DNA, 51 bp, Homo sapiens
tgccattatcttcaaagacttaattgacaatatttgtcacttgcctatgca

SEQ ID NO: 23 - DNA, 51 bp, Homo sapiens
tgccattatcttcaaagacttaattracaatatttgtcacttgcctatgca

SEQ ID NO: 24 - DNA, 51 bp, Homo sapiens
tgccattatcttcaaagacttaattaacaatatttgtcacttgcctatgca

SEQ ID NO: 25 - DNA, 51 bp, Homo sapiens
gcaggctaaggctcagcagtttgggctccaagatgaaaacagcatgtatga

SEQ ID NO: 26 - DNA, 51 bp, Homo sapiens
gcaggctaaggctcagcagtttgggmtccaagatgaaaacagcatgtatga

SEQ ID NO: 27 - DNA, 51 bp, Homo sapiens
gcaggctaaggctcagcagtttgggatccaagatgaaaacagcatgtatga

SEQ ID NO: 28 - DNA, 51 bp, Homo sapiens
cacataaggtcttgtgatgagaattgtaactgttgttgtggctgagttttc

SEQ ID NO: 29 - DNA, 51 bp, Homo sapiens
cacataaggtcttgtgatgagaattrtaactgttgttgtggctgagttttc

SEQ ID NO: 30 - DNA, 51 bp, Homo sapiens
cacataaggtcttgtgatgagaattataactgttgttgtggctgagttttc